SEQUENCE LISTING (3 sequences)

SEQ ID NO: 1 - DNA, 20 bp, Artificial Sequence
  OR1A1 gRNA
gctgaccagtaactcccagg

SEQ ID NO: 2 - DNA, 20 bp, Artificial Sequence
  PRDM1 gRNA
ttggacagatctattccaga

SEQ ID NO: 3 - DNA, 20 bp, Artificial Sequence
  TNFRSF4 gRNA
ggatgtgcgtgggggctcgg

(19) United States
(12) Patent Application Publication
(10) Pub. No.: US 2022/0110974 A1
(43) Pub. Date: Apr. 14, 2022

(54) GENE-REGULATING COMPOSITIONS AND METHODS FOR IMPROVED IMMUNOTHERAPY

(71) Applicant: KSQ Therapeutics, Inc., Cambridge, MA (US)

(72) Inventors: John CHO, Stoneham, MA (US); Jason MERKIN, Watertown, MA (US); Noah Jacob TUBO, Sutton, MA (US); James Martin KABERNA, II, San Francisco, CA (US); Solomon Martin SHENKER, Belmont, MA (US); Kerem Jonatan TUNCEL, Brookline, MA (US)

(21) Appl. No.: 17/426,059

(22) PCT Filed: Jan. 31, 2020
(86) PCT No.: PCT/US2020/016240

§ 371 Date: Jul. 27, 2021

Related U.S. Application Data
(60) Provisional application No. 62/800,121, filed on Feb. 1, 2019.
(60) Provisional application No. 62/916,988, filed on Oct. 18, 2019.

(51) Int. Cl. A61K 35/17; C12N 15/113; C12N 9/22; C07K 14/705; C07K 14/47; A61P 37/00

(52) U.S. Cl. CPC A61K 35/17; C12N 15/1138; A61P 37/00; C07K 14/70578; C07K 14/4703; C12N 9/22

(57) ABSTRACT

The present disclosure provides methods and compositions related to the modification of Tregs to increase therapeutic efficacy. In some embodiments, Tregs modified to reduce expression of one or more endogenous target genes, or to reduce one or more functions of an endogenous protein to enhance immunosuppressive functions of the immune cells are provided. In some embodiments, Tregs further modified by introduction of transgenes conferring antigen specificity, such as exogenous T cell receptors (TCRs) or chimeric antigen receptors (CARs) are provided. Methods of treating an autoimmune diseases using the modified Tregs described herein are also provided.

### FIELD

The disclosure relates to methods, compositions, and components for editing a target nucleic acid sequence, or modulating expression of a target nucleic acid sequence, and applications thereof in connection with immunotherapy, including use with regulatory T cells (optionally receptor engineered regulator T cells), in the treatment of autoimmune diseases.

### BACKGROUND

Inappropriate or exaggerated responses of the immune system cause various symptoms for affected organisms, including autoimmune disorders. Adoptive cell transfer utilizing genetically modified T cells, in particular CAR-T cells has entered clinical testing as a therapeutic for solid and hematologic malignancies. And, adoptive cell transfer has the potential for utility in disorders other than cancer, such as autoimmune disorders. However, factors limiting the efficacy of genetically modified immune cells as include (1) cell proliferation, e.g., limited proliferation of T cells following adoptive transfer; (2) cell survival, e.g., induction of T cell apoptosis; and (3) cell function, e.g., inhibition of T cell function by inhibitory factors and exhaustion of immune cells during manufacturing processes and/or after transfer. There is considerable room for growth in the utilization of adoptive T cells particularly in the treatment of autoimmune disorders, and there exists a need to improve the efficacy of adoptive transfer of modified immune cells in autoimmune disorder treatment.

### SUMMARY

One aspect of the invention disclosed herein relates to a regulatory T cell (Treg) comprising a gene-regulating system capable of reducing expression and/or function of one or more endogenous target genes comprising TNFRSF4, wherein the reduced expression and/or function of the one or more endogenous genes enhances an immunosuppressive function of the Treg. One aspect of the invention disclosed herein relates to a modified Treg wherein the expression and/or function of one or more endogenous target genes comprising TNFRSF4 has been reduced by a gene-regulating system, and wherein the reduced expression and/or function of the one or more endogenous genes enhances an immunosuppressive function of the Treg.

One aspect of the invention disclosed herein relates to a modified Treg comprising a gene-regulating system capable of reducing expression and/or function of one or more endogenous target genes comprising PRDM1, wherein the reduced expression and/or function of the one or more endogenous genes enhances an immunosuppressive function of the Treg. One aspect of the invention disclosed herein relates to a modified Treg wherein the expression and/or function of one or more endogenous target genes comprising PRDM1 has been reduced by a gene-regulating system, and wherein the reduced expression and/or function of the of the one or more endogenous genes enhances an immunosuppressive function of the Treg.

One aspect of the invention disclosed herein relates to a modified Treg comprising a gene-regulating system capable of reducing the expression and/or function of one or more endogenous target genes selected from the group consisting of TNFRSF4, PRDM1, REEP3, MRPL32, FSCN3, KLC3, C4BPA, LZTS1, CDK16, and ADNP, wherein the reduced expression and/or function of the one or more endogenous genes enhances an immunosuppressive function of the Treg. One aspect of the invention disclosed herein relates to a modified Treg wherein the expression and/or function of one or more endogenous target genes selected from the group consisting of TNFRSF4, PRDM1, REEP3, MRPL32, FSCN3, KLC3, C4BPA, LZTS1, CDK16, and ADNP has been reduced by a gene-regulating system, and wherein the reduced expression and/or function of the of the one or more endogenous genes enhances an immunosuppressive function of the Treg.

In certain embodiments, the gene-regulating system comprises (i) a nucleic acid molecule; (ii) an enzymatic protein; or (iii) a nucleic acid molecule and an enzymatic protein. In an embodiment, the gene-regulating system comprises a nucleic acid molecule selected from an siRNA, an shRNA, a microRNA (miR), an antagomiR, or an antisense RNA. In one embodiment, the gene-regulating system comprises an enzymatic protein, and wherein the enzymatic protein has been engineered to specifically bind to a target sequence in one or more of the endogenous genes. In some embodiments, the protein is a Transcription activator-like effector nuclease (TALEN), a zinc-finger nuclease, or a meganuclease.

In an embodiment, the gene-regulating system comprises a nucleic acid molecule and an enzymatic protein, wherein the nucleic acid molecule is a guide RNA (gRNA) molecule and the enzymatic protein is a Cas protein or Cas ortholog. In one embodiment, the Cas protein is a Cas9 protein. In some embodiments, the Cas protein is a wild-type Cas protein comprising two enzymatically active domains, and capable of inducing double stranded DNA breaks. In embodiments, the Cas protein is a Cas nickase mutant comprising one enzymatically active domain and capable of inducing single stranded DNA breaks. In an embodiment, the Cas protein is a deactivated Cas protein (dCas) and is associated with a heterologous protein capable of modulating the expression of the one or more endogenous target genes. In embodiments, the heterologous protein is selected from the group consisting of MAX-interacting protein 1 (MXI1), Krüppel-associated box (KRAB) domain, methyl-CpG binding protein 2 (MECP2), and four concatenated mSin3 domains (SID4X).

In embodiments, the gene-regulating system is capable of reducing the expression and/or function of at least 2, 3, 4, 5, 6 or more of endogenous target genes.

In embodiments, the gene-regulating system is capable of reducing the expression and/or function of a plurality of endogenous target genes selected from the group consisting of TNFRSF4, PRDM1, REEP3, MRPL32, FSCN3, KLC3, C4BPA, LZTS1, CDK16, and ADNP. In some embodiments, the gene-regulating system is capable of reducing the expression and/or function of at least 2, 3, 4, 5, 6 or more of endogenous target genes selected from the group consisting of TNFRSF4, PRDM1, REEP3, MRPL32, FSCN3, KLC3, C4BPA, LZTS1, CDK16, and ADNP.

In an embodiment, the gene-regulating system is capable of reducing the expression and/or function of a plurality of endogenous target genes, wherein at least one of the plurality of target genes is TNFRSF4 and wherein at least one of the plurality of target genes is selected from PRDM1, REEP3, MRPL32, FSCN3, KLC3, C4BPA, LZTS1, CDK16, and ADNP. In an embodiment, one of the plurality of target genes is TNFRSF4 and wherein at least 2, 3, 4, 5, 6 or more of the plurality of target genes are selected from PRDM1, REEP3, MRPL32, FSCN3, KLC3, C4BPA, LZTS1, CDK16, and ADNP.

In one embodiment, the gene-regulating system is capable of reducing the expression and/or function of a plurality of endogenous target genes, wherein at least one of the plurality of target genes is PRDM1 and wherein at least one of the plurality of target genes is selected from TNFRSF4, REEP3, MRPL32, FSCN3, KLC3, C4BPA, LZTS1, CDK16, and ADNP. In one embodiment, one of the plurality of target genes is PRDM1 and wherein at least 2, 3, 4, 5, 6 or more of the plurality of target genes are selected from TNFRSF4, REEP3, MRPL32, FSCN3, KLC3, C4BPA, LZTS1, CDK16, and ADNP.

In some embodiments, the gene-regulating system comprises a plurality of gRNA molecules. In other embodiments, the gene-regulating system is introduced to the Treg by transfection, transduction, electroporation, or physical disruption of the cell membrane by a microfluidics device. In embodiments, the gene-regulating system is introduced as a polynucleotide encoding one or more components of the system, a protein, or a ribonucleoprotein (RNP) complex.

In certain embodiments, the immunosuppressive function is selected from Treg proliferation, Treg viability, Treg stability, increased expression or secretion of an immunosuppressive cytokine, optionally wherein the immunosuppressive cytokine is IL-10, increased co-expression of Foxp3 and Helios, and/or resistance to exhaustion. In one embodiment, the modified Treg further comprises an engineered immune receptor displayed on the cell surface. In embodiments, the engineered immune receptor is a chimeric antigen receptor (CAR) comprising an antigen-binding domain, a transmembrane domain, and an intracellular signaling domain.

In certain embodiments, the engineered immune receptor is an engineered T cell receptor (TCR). In an embodiment, the engineered immune receptor specifically binds to an antigen expressed on a target cell.

One aspect of the invention disclosed herein relates to a modified Treg comprising reduced expression and/or function of one or more endogenous genes relative to the expression and/or function of the one or more endogenous genes in a non-modified Treg, wherein the one more endogenous genes comprises TNFRSF4, and wherein the reduced expression and/or function of the one or more endogenous genes enhances an immunosuppressive function of the Treg.

One aspect of the invention disclosed herein relates to a modified Treg comprising reduced expression and/or function of one or more endogenous genes relative to the expression and/or function of the one or more endogenous genes in a non-modified Treg, wherein the one more endogenous genes comprises PRDM1, and wherein the reduced expression and/or function of the one or more endogenous genes enhances an immunosuppressive function of the Treg.

One aspect of the invention disclosed herein relates to a modified Treg comprising reduced expression and/or function of one or more endogenous genes relative to the expression and/or function of the one or more endogenous genes in a non-modified Treg, wherein the one or more endogenous genes are selected from the group consisting of TNFRSF4, PRDM1, REEP3, MRPL32, FSCN3, KLC3, C4BPA, LZTS1, CDK16, and ADNP, and wherein the reduced expression and/or function of the one or more endogenous genes enhances an immunosuppressive function of the Treg.

In some embodiments, the modified Treg further comprises an engineered immune receptor displayed on the cell surface. In embodiments, the engineered immune receptor is a CAR or an engineered TCR. In one embodiment, the engineered immune receptor specifically binds to an antigen expressed on a target cell.

In one embodiment, the modified Treg further comprises reduced expression of TNFRSF4. In one embodiment, the modified Treg comprises reduced expression and/or function of TNFRSF4 and reduced expression and/or function of at least one target gene selected from PRDM1, REEP3, MRPL32, FSCN3, KLC3, C4BPA, LZTS1, CDK16, and ADNP. In an embodiment, the modified Treg comprises reduced expression and/or function of TNFRSF4 and reduced expression and/or function of at least 2, 3, 4, 5, 6 or more target genes selected from the group consisting of PRDM1, REEP3, MRPL32, FSCN3, KLC3, C4BPA, LZTS1, CDK16, and ADNP.

In one embodiment, the modified Treg further comprises reduced expression of PRDM1. In an embodiment, the modified Treg comprises reduced expression and/or function of PRDM1 and reduced expression and/or function of at least one target gene selected from TNFRSF4, REEP3, MRPL32, FSCN3, KLC3, C4BPA, LZTS1, CDK16, and ADNP. In one embodiment, the modified Treg comprises reduced expression and/or function of PRDM1 and reduced expression and/or function of at least 2, 3, 4, 5, 6 or more target genes selected from the group consisting of TNFRSF4, REEP3, MRPL32, FSCN3, KLC3, C4BPA, LZTS1, CDK16, and ADNP.

In embodiments, the gene-regulating system comprises a nucleic acid molecule selected from an siRNA and an shRNA. In certain embodiments, the gene-regulating system is further capable of reducing the expression of one or more endogenous target genes selected from the group consisting of TNFRSF4, PRDM1, REEP3, MRPL32, FSCN3, KLC3, C4BPA, LZTS1, CDK16, and ADNP. In an embodiment, the gene-regulating system is capable of reducing the expression and/or function of a plurality of endogenous target genes and comprises a plurality of siRNAs or shRNAs, wherein at least one endogenous target gene is selected from the group consisting of TNFRSF4, PRDM1, REEP3, MRPL32, FSCN3, KLC3, C4BPA, LZTS1, CDK16, and ADNP.

In certain embodiments, the gene-regulating system is capable of reducing the expression and/or function of at least 2, 3, 4, 5, 6 or more of endogenous target genes selected from the group consisting of TNFRSF4, PRDM1, REEP3, MRPL32, FSCN3, KLC3, C4BPA, LZTS1, CDK16, and ADNP. In an embodiment, the gene-regulating system is capable of reducing the expression and/or function of a plurality of endogenous target genes and comprises a plurality of siRNAs or shRNAs, wherein at least one of the plurality of target genes is TNFRSF4 and at least one of the plurality of target genes is selected from PRDM1, REEP3, MRPL32, FSCN3, KLC3, C4BPA, LZTS1, CDK16, and ADNP2. In certain embodiments, at least one of the plurality of target genes is TNFRSF4 and at least at least 2, 3, 4, 5, 6 or more of the plurality of target genes are selected from PRDM1, REEP3, MRPL32, FSCN3, KLC3, C4BPA, LZTS1, CDK16, and ADNP. In another embodiment, the gene-regulating system is capable of reducing the expression and/or function of a plurality of endogenous target genes and comprises a plurality of siRNAs or shRNAs, wherein at least one of the plurality of target genes is PRDM1 and at least one of the plurality of target genes is selected from TNFRSF4, REEP3, MRPL32, FSCN3, KLC3, C4BPA, LZTS1, CDK16, and ADNP2. In one embodiment, at least one of the plurality of target genes is PRDM1 and at least at least 2, 3, 4, 5, 6 or more of the plurality of target genes are selected from TNFRSF4, REEP3, MRPL32, FSCN3, KLC3, C4BPA, LZTS1, CDK16, and ADNP.

One aspect of the invention disclosed herein relates to a composition comprising a modified Treg disclosed herein. In an embodiment, the composition comprises at least 1×10⁴, 1×10⁵, 1×10⁶, 1×10⁷, 1×10⁸, 1×10⁹, or 1×10¹⁰ modified Tregs. In certain embodiments, the composition is suitable for administration to a subject in need thereof. In some embodiments, the composition comprises autologous Tregs derived from the subject in need thereof. In an embodiment, the composition comprises allogeneic Tregs derived from a donor subject.

One aspect of the invention disclosed herein relates to a gene-regulating system capable of reducing expression of one or more endogenous target genes in a cell, wherein the system comprises (i) a nucleic acid molecule; (ii) an enzymatic protein; or (iii) a nucleic acid molecule and an enzymatic protein, and wherein the one or more endogenous target genes comprises TNFRSF4. In embodiments, the system comprises a guide RNA (gRNA) nucleic acid molecule and a Cas endonuclease.

One aspect of the invention disclosed herein relates to a gene-regulating system capable of reducing expression of one or more endogenous target genes in a cell, wherein the system comprises (i) a nucleic acid molecule; (ii) an enzymatic protein; or (iii) a nucleic acid molecule and an enzymatic protein, and wherein the one or more endogenous target genes comprises PRDM1. In some embodiments, the system comprises a guide RNA (gRNA) nucleic acid molecule and a Cas endonuclease.

One aspect of the invention disclosed herein relates to a gene-regulating system capable of reducing expression and/or function of one or more endogenous target genes in a cell, wherein the system comprises (i) a nucleic acid molecule; (ii) an enzymatic protein; or (iii) a nucleic acid molecule and an enzymatic protein, and wherein the one or more endogenous target genes are selected from the group consisting REEP3, MRPL32, FSCN3, KLC3, C4BPA, LZTS1, CDK16, and ADNP2. In embodiments, the system comprises a guide RNA (gRNA) nucleic acid molecule and a Cas endonuclease.

Any Cas protein, including those provided herein, can be used. In embodiments, the Cas protein is a Cas9 protein. In some embodiments, the Cas protein is a wild-type Cas protein comprising two enzymatically active domains, and capable of inducing double stranded DNA breaks. In certain embodiments, the Cas protein is a Cas nickase mutant comprising one enzymatically active domain and capable of inducing single stranded DNA breaks. In some embodiments, the Cas protein is a deactivated Cas protein (dCas) and is associated with a heterologous protein capable of modulating the expression of the one or more endogenous target genes.

In an embodiment, the heterologous protein is selected from the group consisting of MAX-interacting protein 1 (MXI1), Krüppel-associated box (KRAB) domain, and four concatenated mSin3 domains (SID4X).

In one embodiment, the system comprises a nucleic acid molecule and wherein the nucleic acid molecule is an siRNA, an shRNA, a microRNA (miR), an antagomiR, or an antisense RNA.

In some embodiments, the system comprises a protein comprising a DNA binding domain and an enzymatic domain and is selected from a zinc finger nuclease and a transcription-activator-like effector nuclease (TALEN).

One aspect of the invention disclosed herein relates to a kit comprising a gene-regulating system disclosed herein.

One aspect of the invention disclosed herein relates to a gRNA nucleic acid molecule comprising a targeting domain nucleic acid sequence that is complementary to a target sequence in an endogenous target gene, wherein the endogenous target gene is TNFRSF4. One aspect of the invention disclosed herein relates to a gRNA nucleic acid molecule comprising a targeting domain nucleic acid sequence that is complementary to a target sequence in an endogenous target gene, wherein the endogenous target gene is PRDM1. One aspect of the invention disclosed herein relates to a gRNA nucleic acid molecule comprising a targeting domain nucleic acid sequence that is complementary to a target sequence in an endogenous target gene, wherein the endogenous target gene is selected from REEP3, MRPL32, FSCN3, KLC3, C4BPA, LZTS1, CDK16, and ADNP2.

In embodiments, the target sequence comprises a PAM sequence. In certain embodiments, the gRNA is a modular gRNA molecule. In an embodiment, the gRNA is a dual gRNA molecule. In some embodiments, the targeting domain is 16, 17, 18, 19, 20, 21, 22, 23, 24, 25, 26 or more nucleotides in length. In an embodiment, the gRNA molecule comprises a modification at or near its 5′ end (e.g., within 1-10, 1-5, or 1-2 nucleotides of its 5′ end) and/or a modification at or near its 3′ end (e.g., within 1-10, 1-5, or 1-2 nucleotides of its 3′ end).

In some embodiments, the modified gRNA exhibits increased stability towards nucleases when introduced into a T cell. In some embodiments, the modified gRNA exhibits a reduced innate immune response when introduced into a T cell.

One aspect of the invention disclosed herein relates to a polynucleotide molecule encoding a gRNA molecule disclosed herein. One aspect of the invention disclosed herein relates to a polynucleotide molecule encoding a plurality of gRNA molecules disclosed herein.

One aspect of the invention disclosed herein relates to a composition comprising one or more gRNA molecules disclosed herein or a polynucleotide disclosed herein. One aspect of the invention disclosed herein relates to a kit comprising a gRNA molecule disclosed herein or a polynucleotide disclosed herein.

One aspect of the invention disclosed herein relates to a method of producing a modified Treg comprising: obtaining an Treg from a subject; introducing a gene-regulating system into the Treg, wherein the gene-regulating system is capable of reducing expression and/or function of one or more endogenous target genes, and wherein the one or more endogenous target genes comprises TNFRSF4; and culturing the Treg such that the expression and/or function of one or more endogenous target genes is reduced compared to an Treg that has not been modified.

One aspect of the invention disclosed herein relates to a method of producing a modified Treg comprising: obtaining a Treg from a subject; introducing a gene-regulating system into the Treg, wherein the gene-regulating system is capable of reducing expression and/or function of one or more endogenous target genes, and wherein the one or more endogenous target genes comprises PRDM1; and culturing the Treg such that the expression and/or function of one or more endogenous target genes is reduced compared to a Treg that has not been modified.

One aspect of the invention disclosed herein relates to a method of producing a modified Treg comprising: introducing a gene-regulating system into the Treg, wherein the gene-regulating system is capable of reducing expression and/or function of one or more endogenous target genes, wherein the one or more endogenous target genes comprises TNFRSF4. One aspect of the invention disclosed herein relates to a method of producing a modified Treg comprising: introducing a gene-regulating system into the Treg, wherein the gene-regulating system is capable of reducing expression and/or function of one or more endogenous target genes, wherein the one or more endogenous target genes comprises PRDM1.

In embodiments, the gene-regulating system is any system disclosed herein. In embodiments, the method further comprises introducing a polynucleotide sequence encoding an engineered immune receptor selected from a CAR and a TCR. In embodiments, the gene-regulating system and/or the polynucleotide encoding the engineered immune receptor are introduced to the Treg by transfection, transduction, electroporation, or physical disruption of the cell membrane by a microfluidics device. In an embodiment, the gene-regulating system is introduced as a polynucleotide sequence encoding one or more components of the system, as a protein, or as an ribonucleoprotein (RNP) complex.

One aspect of the invention disclosed herein relates to a method of producing a modified Treg comprising: obtaining a population of Tregs; expanding the population of Tregs; and introducing a gene-regulating system into the population of Tregs, wherein the gene-regulating system is capable of reducing expression and/or function of one or more endogenous target genes comprising TNFRSF4. In embodiments, the gene-regulating system is introduced to the population of Tregs prior to the expansion. In an embodiment, the gene-regulating system is introduced to the population of Tregs after the expansion.

One aspect of the invention disclosed herein relates to a method of producing a modified Treg comprising: obtaining a population of Tregs; expanding the population of Tregs; and introducing a gene-regulating system into the population of Tregs, wherein the gene-regulating system is capable of reducing expression and/or function of one or more endogenous target genes comprising PRDM1. In an embodiment, the gene-regulating system is introduced to the population of Tregs prior to expansion. In embodiments, the gene-regulating system is introduced to the population of Tregs after the expansion.

One aspect of the invention disclosed herein relates to a method of treating a disease or disorder in a subject in need thereof comprising administering an effective amount of a modified Treg disclosed herein, or a composition disclosed herein.

In embodiments, the disease or disorder is an autoimmune disorder. In embodiments, the autoimmune disorder is autoimmune hepatitis, inflammatory bowel disease (IBD), Crohn's disease, colitis, ulcerative colitis, type 1 diabetes, alopecia areata, vasculitis, temporal arthritis, lupus, celiac disease, Sjogrens syndrome, polymyalgia rheumatica, multiple sclerosis, arthritis, rheumatoid arthritis, graft versus host disease (GVHD), or psoriasis. In certain embodiments, the autoimmune disorder is an inflammatory bowel disease (IBD), e.g., Crohn's disease or ulcerative colitis. In certain embodiments, the autoimmune disorder is systemic lupus erythematosus. In certain embodiments, the autoimmune disorder is an autoimmune response associated with a solid organ transplant, e.g., GVHD. In certain embodiments, the modified Tregs are autologous to the subject. In an embodiment, the modified Tregs are allogenic to the subject.

One aspect of the invention disclosed herein relates to a method of enhancing one or more immunosuppressive function of a Treg comprising: introducing a gene-regulating system into the Treg, wherein the gene-regulating system is capable of reducing the expression and/or function of one or more endogenous target genes, and wherein the one or more endogenous target genes comprises TNFRSF4; and culturing the Treg such that the expression and/or function of one or more endogenous target genes is reduced compared to a Treg that has not been modified, wherein the modified Treg demonstrates one or more enhanced immunosuppressive functions compared to the Treg that has not been modified.

One aspect of the invention disclosed herein relates to a method of enhancing one or more immunosuppressive functions of a Treg comprising: introducing a gene-regulating system into the Treg, wherein the gene-regulating system is capable of reducing the expression and/or function of one or more endogenous target genes, and wherein the one or more endogenous target genes comprises PRDM1; and culturing the Treg such that the expression and/or function of one or more endogenous target genes is reduced compared to a Treg that has not been modified, wherein the modified Treg demonstrates one or more enhanced immunosuppressive functions compared to the Treg that has not been modified.

One aspect of the invention disclosed herein relates to a method of enhancing one or more immunosuppressive functions of a Treg comprising: introducing a gene-regulating system into the Treg, wherein the gene-regulating system is capable of reducing the expression and/or function of one or more endogenous target genes, wherein the one or more endogenous target genes comprises TNFRSF4.

One aspect of the invention disclosed herein relates to a method of enhancing one or more immunosuppressive functions of a Treg comprising: introducing a gene-regulating system into the Treg, wherein the gene-regulating system is capable of reducing the expression and/or function of one or more endogenous target genes, wherein the one or more endogenous target genes comprises PRDM1.

In embodiments, the one or more immunosuppressive functions are selected from Treg proliferation, Treg viability, Treg stability, increased expression or secretion of an immunosuppressive cytokine, optionally wherein the immunosuppressive cytokine is IL-10, increased co-expression of Foxp3 and Helios, and/or resistance to exhaustion.

One aspect of the invention disclosed herein relates to a method of enhancing one or more immunosuppressive functions of a Treg comprising: introducing a gene-regulating system into the Treg, wherein the gene-regulating system is capable of reducing the expression and/or function of one or more endogenous target genes, wherein the one or more endogenous target genes comprises TNFRSF4 and wherein the introduction of the gene-regulating system does not decrease the stability of the Treg. Stability of the Treg can be assessed, for example, by measuring the methylation of Foxp3 TSDR.

One aspect of the invention disclosed herein relates to a method of enhancing one or more immunosuppressive functions of a Treg comprising: introducing a gene-regulating system into the Treg, wherein the gene-regulating system is capable of reducing the expression and/or function of one or more endogenous target genes, wherein the one or more endogenous target genes comprises PRDM1 and wherein the introduction of the gene-regulating system increases the stability of the Treg. Stability of the Treg can be assessed, for example, by measuring the methylation of Foxp3 TSDR.

Thus, for example, the introduction of the gene-regulating system can increase the percentage of demethylated Foxp3 TSDR by at least 10%, by at least 15%, by at least 20%, or by at least 25%. The introduction of the gene-regulating system can increase the percentage of demethylated Foxp3 TSDR by 10-50% 10-30%, 15-50%, 15-30% 20-50%, 20-30%, 25-50%, or 25-30%.

One aspect of the invention disclosed herein relates to a method of treating an autoimmune disease in a subject in need thereof comprising administering an effective amount of a modified Treg disclosed herein, or the composition disclosed herein. In embodiments, the autoimmune disease is selected from the group consisting of: autoimmune hepatitis, inflammatory bowel disease (IBD), Crohn's disease, colitis, ulcerative colitis, type 1 diabetes, alopecia areata, vasculitis, temporal arthritis, lupus, celiac disease, Sjogrens syndrome, polymyalgia rheumatica, multiple sclerosis, arthritis, rheumatoid arthritis, graft versus host disease (GVHD), and psoriasis. In certain embodiments, the autoimmune disorder is an inflammatory bowel disease (IBD), e.g., Crohn's disease or ulcerative colitis. In certain embodiments, the autoimmune disorder is systemic lupus erythematosus.

One aspect of the invention disclosed herein relates to a method of treating an autoimmune response associated with solid organ transplant, e.g., GVHD, in a subject in need thereof comprising administering an effective amount of a modified Treg disclosed herein, or the composition disclosed herein.

In some aspects, the modified Treg is a tissue-resident Treg. In some aspects, the Treg is a tissue-resident Treg.

### BRIEF DESCRIPTION OF THE FIGURES

FIG. 1 summarizes the Treg-selective targets identified through in vitro CRISPR/Cas9 functional genomic screen

FIG. 2A and FIG. 2B illustrate editing of the Foxp3 and CD45 genes in human Treg cells using methods described herein.

FIG. 3A and FIG. 3B demonstrate improved proliferative capacity of PRDM1- and TNFRSF-edited Treg cells in an in vitro culture system.

FIG. 4A and FIG. 4B demonstrate increase proportion of Foxp3⁺Helios⁺ cells in PRDM1-edited Treg cells in an in vitro culture system.

FIG. 5 demonstrates that Foxp3 Treg-specific demethylated region (TSDR) demethylation, a measure of Treg stability, is maintained in TNFRSF4-edited Treg cells and is increased is PRDM1-edited T reg cells.

FIG. 6A and FIG. 6B demonstrate increased production of the immunosuppressive cytokine interleukin-10 in PRDM1- and TNFRSF-edited Treg cells in an in vitro culture system.

FIG. 7A and FIG. 7B demonstrate that PRDM1-edited Treg cells persist under inflammatory conditions.

FIG. 8 demonstrates that the suppressive capacities of PRDM1- and TNFRSF4-edited Tregs are comparable to that of control-edited Tregs.

FIG. 9A demonstrates that the treatment of mice with PRDM1- and TNFRSF4-edited Tregs exhibit enhanced survival versus mice treated with control-edited Tregs in a model of GvHD.

FIG. 9B demonstrates reduced proliferative capacity of CD8+ effector T cells as a consequence of Treg treatment.

### DETAILED DESCRIPTION

The present disclosure provides methods and compositions related to the modification of T regulatory cells (Treg) to increase their therapeutic efficacy in the context of immunotherapy for autoimmune diseases. In some embodiments, Tregs are modified by the methods of the present disclosure to reduce expression of one or more endogenous target genes, or to reduce one or more functions of an endogenous protein such that one or more immunosuppressive functions of the immune cells are enhanced. In some embodiments, the Tregs are further modified by introduction of transgenes conferring antigen specificity, such as introduction of T cell receptor (TCR) or chimeric antigen receptor (CAR) expression constructs. In some embodiments, the present disclosure provides compositions and methods for modifying Tregs, such as compositions of gene-regulating systems. In some embodiments, the present disclosure provides methods of treating an autoimmune disorder, comprising administration of the modified Tregs described herein to a subject in need thereof.

### I. Definitions

As used in this specification and the appended claims, the singular forms “a,” “an” and “the” include plural references unless the content clearly dictates otherwise.

As used in this specification, the term “and/or” is used in this disclosure to mean either “and” or “or” unless indicated otherwise.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Decrease” or “reduce” refers to a decrease or a reduction in a particular value of at least 5%, for example, a 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% decrease as compared to a reference value. A decrease or reduction in a particular value may also be represented as a fold-change in the value compared to a reference value, for example, at least a 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold, or more, decrease as compared to a reference value.

“Increase” refers to an increase in a particular value of at least 5%, for example, a 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, 100%, 200%, 300%, 400%, 500%, or more increase as compared to a reference value. An increase in a particular value may also be represented as a fold-change in the value compared to a reference value, for example, at least a 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more, increase as compared to the level of a reference value.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

“Fragment” refers to a portion of a polypeptide or polynucleotide molecule containing less than the entire polypeptide or polynucleotide sequence. In some embodiments, a fragment of a polypeptide or polynucleotide comprises at least 10%, 20%, 30%, 40%, 50%, 60%, 70%, 80%, 90%, 95%, 96%, 97%, 98%, or 99% of the entire length of the reference polypeptide or polynucleotide. In some embodiments, a polypeptide or polynucleotide fragment may contain 5, 10, 15, 20, 25, 30, 35, 40, 45, 50, 60, 70, 80, 90, 100, 200, 300, 400, 500, 600, 700, 800, 900, 1000, or more nucleotides or amino acids.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

“Complementary” refers to the capacity for pairing, through base stacking and specific hydrogen bonding, between two sequences comprising naturally or non-naturally occurring bases or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

As referred to herein, a “complementary nucleic acid sequence” is a nucleic acid sequence comprising a sequence of nucleotides that enables it to non-covalently bind to another nucleic acid in a sequence-specific, antiparallel, manner (i.e., a nucleic acid specifically binds to a complementary nucleic acid) under the appropriate in vitro and/or in vivo conditions of temperature and solution ionic strength.

Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

Herein, the term “hybridize” refers to pairing between complementary nucleotide bases (e.g., adenine (A) forms a base pair with thymine (T) in a DNA molecule and with uracil (U) in an RNA molecule, and guanine (G) forms a base pair with cytosine (C) in both DNA and RNA molecules) to form a double-stranded nucleic acid molecule. (See, e.g., Wahl and Berger (1987) Methods Enzymol. 152:399; Kimmel, (1987) Methods Enzymol. 152:507). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein-binding segment (dsRNA duplex) of a guide RNA molecule, the position is not considered to be non-complementary, but is instead considered to be complementary. It is understood in the art that the sequence of polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted.

The term “modified” refers to a substance or compound (e.g., a cell, a polynucleotide sequence, and/or a polypeptide sequence) that has been altered or changed as compared to the corresponding unmodified substance or compound.

The term “naturally-occurring” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

“Isolated” refers to a material that is free to varying degrees from components which normally accompany it as found in its native state.

An “expression cassette” or “expression construct” refers to a DNA polynucleotide sequence operably linked to a promoter. “Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

The term “recombinant vector” as used herein refers to a polynucleotide molecule capable transferring or transporting another polynucleotide inserted into the vector. The inserted polynucleotide may be an expression cassette. In some embodiments, a recombinant vector may be viral vector or a non-viral vector (e.g., a plasmid).

The term “sample” refers to a biological composition (e.g., a cell or a portion of a tissue) that is subjected to analysis and/or genetic modification. In some embodiments, a sample is a “primary sample” in that it is obtained directly from a subject; in some embodiments, a “sample” is the result of processing of a primary sample, for example to remove certain components and/or to isolate or purify certain components of interest.

The term “subject” includes animals, such as e.g. mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

“Administration” refers herein to introducing an agent or composition into a subject.

“Treating” as used herein refers to delivering an agent or composition to a subject to affect a physiologic outcome.

As used herein, the term “effective amount” refers to the minimum amount of an agent or composition required to result in a particular physiological effect. The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (EC₅₀), which refers to the concentration of an agent that results in a magnitude of a particular physiological response that is half-way between a reference level and a maximum response level.

“Population” of cells refers to any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, at least 1×10¹⁰ cells, or more cells. A population of cells may refer to an in vitro population (e.g., a population of cells in culture) or an in vivo population (e.g., a population of cells residing in a particular tissue).

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### II. Modified Regulatory T Cells

In some embodiments, the present disclosure provides modified T regulatory cells (Tregs). Herein, the term “modified Tregs” encompasses Treg cells comprising one or more genomic modifications resulting in the reduced expression and/or function of one or more endogenous target genes as well as Tregs comprising a gene-regulating system capable of reducing the expression and/or function of one or more endogenous target genes. Herein, an “un-modified Treg” or “control Treg” refers to a cell or population of cells wherein the genomes have not been modified and that does not comprise a gene-regulating system or comprises a control gene-regulating system (e.g., an empty vector control, a non-targeting gRNA, a scrambled siRNA, etc.). In some embodiments, the Treg or the modified Treg can be a tissue-resident Treg.

In some embodiments, the modified Treg is an animal cell or is derived from an animal cell, including invertebrate animals and vertebrate animals (e.g., fish, amphibian, reptile, bird, or mammal). In some embodiments, the modified Treg is a mammalian cell or is derived from a mammalian cell (e.g., a pig, a cow, a goat, a sheep, a rodent, a non-human primate, a human, etc.). In some embodiments, the modified Treg is a rodent cell or is derived from a rodent cell (e.g., a rat or a mouse). In some embodiments, the modified Treg is a human cell or is derived from a human cell.

In some embodiments, the modified Tregs comprise one or more modifications (e.g., insertions, deletions, or mutations of one or more nucleic acids) in the genomic DNA sequence of an endogenous target gene resulting in the reduced expression and/or function the endogenous gene. In such embodiments, the modified Tregs comprise a “modified endogenous target gene.” In some embodiments, the modifications in the genomic DNA sequence reduce or inhibit mRNA transcription, thereby reducing the expression level of the encoded mRNA transcript and protein. In some embodiments, the modifications in the genomic DNA sequence reduce or inhibit mRNA translation, thereby reducing the expression level of the encoded protein. In some embodiments, the modifications in the genomic DNA sequence encode a modified endogenous protein with reduced or altered function compared to the unmodified (i.e., wild-type) version of the endogenous protein (e.g., a dominant-negative mutant, described infra).

In some embodiments, the modified Tregs comprise one or more genomic modifications at a genomic location other than an endogenous target gene that result in the reduced expression and/or function of the endogenous target gene or that result in the expression of a modified version of an endogenous protein. For example, in some embodiments, a polynucleotide sequence encoding a gene regulating system is inserted into one or more locations in the genome, thereby reducing the expression and/or function of an endogenous target gene upon the expression of the gene-regulating system. In some embodiments, a polynucleotide sequence encoding a modified version of an endogenous protein is inserted at one or more locations in the genome, wherein the function of the modified version of the protein is reduced compared to the un-modified or wild-type version of the protein (e.g., a dominant-negative mutant, described infra).

In some embodiments, the modified Tregs described herein comprise one or more modified endogenous target genes, wherein the one or more modifications result in a reduced expression and/or function of a gene product (i.e., an mRNA transcript or a protein) encoded by the endogenous target gene compared to an unmodified Treg. For example, in some embodiments, a modified Treg demonstrates reduced expression of an mRNA transcript and/or reduced expression of a protein. In some embodiments, the expression of the gene product in a modified Treg is reduced by at least 5% compared to the expression of the gene product in an unmodified Treg. In some embodiments, the expression of the gene product in a modified Treg is reduced by at least 10%, 20%, 30%, 40%, 50%, 60%, 70%, 80%, 90%, or more compared to the expression of the gene product in an unmodified Treg. In some embodiments, the modified Tregs described herein demonstrate reduced expression and/or function of gene products encoded by a plurality (e.g., two or more) of endogenous target genes compared to the expression of the gene products in an unmodified Treg. For example, in some embodiments, a modified Treg demonstrates reduced expression and/or function of gene products from 2, 3, 4, 5, 6, 7, 8, 9, 10, or more endogenous target genes compared to the expression of the gene products in an unmodified Treg.

In some embodiments, the present disclosure provides a modified Treg wherein one or more endogenous target genes, or a portion thereof, are deleted (i.e., “knocked-out”) such that the modified Treg does not express the mRNA transcript or protein. In some embodiments, a modified Treg comprises deletion of a plurality of endogenous target genes, or portions thereof. In some embodiments, a modified Treg comprises deletion of 2, 3, 4, 5, 6, 7, 8, 9, 10, or more endogenous target genes.

In some embodiments, the modified Tregs described herein comprise one or more modified endogenous target genes, wherein the one or more modifications to the target DNA sequence result in expression of a protein with reduced or altered function (e.g., a “modified endogenous protein”) compared to the function of the corresponding protein expressed in an unmodified Treg (e.g., a “unmodified endogenous protein”). In some embodiments, the modified Tregs described herein comprise 2, 3, 4, 5, 6, 7, 8, 9, 10, or more modified endogenous target genes encoding 2, 3, 4, 5, 6, 7, 8, 9, 10, or more modified endogenous proteins. In some embodiments, the modified endogenous protein demonstrates reduced or altered binding affinity for another protein expressed by the modified Treg or expressed by another cell; reduced or altered signaling capacity; reduced or altered enzymatic activity; reduced or altered DNA-binding activity; or reduced or altered ability to function as a scaffolding protein.

In some embodiments, the modified endogenous target gene comprises one or more dominant negative mutations. As used herein, a “dominant-negative mutation” refers to a substitution, deletion, or insertion of one or more nucleotides of a target gene such that the encoded protein acts antagonistically to the protein encoded by the unmodified target gene. The mutation is dominant-negative because the negative phenotype confers genic dominance over the positive phenotype of the corresponding unmodified gene. A gene comprising one or more dominant-negative mutations and the protein encoded thereby are referred to as a “dominant-negative mutants”, e.g. dominant-negative genes and dominant-negative proteins. In some embodiments, the dominant negative mutant protein is encoded by an exogenous transgene inserted at one or more locations in the genome of the Treg.

Various mechanisms for dominant negativity are known. Typically, the gene product of a dominant negative mutant retains some functions of the unmodified gene product but lacks one or more crucial other functions of the unmodified gene product. This causes the dominant-negative mutant to antagonize the unmodified gene product. For example, as an illustrative embodiment, a dominant-negative mutant of a transcription factor may lack a functional activation domain but retain a functional DNA binding domain. In this example, the dominant-negative transcription factor cannot activate transcription of the DNA as the unmodified transcription factor does, but the dominant-negative transcription factor can indirectly inhibit gene expression by preventing the unmodified transcription factor from binding to the transcription-factor binding site. As another illustrative embodiment, dominant-negative mutations of proteins that function as dimers are known. Dominant-negative mutants of such dimeric proteins may retain the ability to dimerize with unmodified protein but be unable to function otherwise. The dominant-negative monomers, by dimerizing with unmodified monomers to form heterodimers, prevent formation of functional homodimers of the unmodified monomers.

In some embodiments, the modified Tregs comprise a gene-regulating system capable of reducing the expression or function of one or more endogenous target genes. The gene-regulating system can reduce the expression and/or function of the endogenous target genes modifications by a variety of mechanisms including by modifying the genomic DNA sequence of the endogenous target gene (e.g., by insertion, deletion, or mutation of one or more nucleic acids in the genomic DNA sequence); by regulating transcription of the endogenous target gene (e.g., inhibition or repression of mRNA transcription); and/or by regulating translation of the endogenous target gene (e.g., by mRNA degradation).

In some embodiments, the modified Tregs described herein comprise a gene-regulating system (e.g., a nucleic acid-based gene-regulating system, a protein-based gene-regulating system, or a combination protein/nucleic acid-based gene-regulating system). In such embodiments, the gene-regulating system comprised in the modified Treg is capable of modifying one or more endogenous target genes. In some embodiments, the modified Tregs described herein comprise a gene-regulating system comprising:

(a) one or more nucleic acid molecules capable of reducing the expression or modifying the function of a gene product encoded by one or more endogenous target genes;

(b) one or more polynucleotides encoding a nucleic acid molecule that is capable of reducing the expression or modifying the function of a gene product encoded by one or more endogenous target genes;

(c) one or more proteins capable of reducing the expression or modifying the function of a gene product encoded by one or more endogenous target genes;

(d) one or more polynucleotides encoding a protein that is capable of reducing the expression or modifying the function of a gene product encoded by one or more endogenous target genes;

(e) one or more guide RNAs (gRNAs) capable of binding to a target DNA sequence in an endogenous gene;

(f) one or more polynucleotides encoding one or more gRNAs capable of binding to a target DNA sequence in an endogenous gene;

(g) one or more site-directed modifying polypeptides capable of interacting with a gRNA and modifying a target DNA sequence in an endogenous gene;

(h) one or more polynucleotides encoding a site-directed modifying polypeptide capable of interacting with a gRNA and modifying a target DNA sequence in an endogenous gene;

(i) one or more guide DNAs (gDNAs) capable of binding to a target DNA sequence in an endogenous gene;

(j) one or more polynucleotides encoding one or more gDNAs capable of binding to a target DNA sequence in an endogenous gene;

(k) one or more site-directed modifying polypeptides capable of interacting with a gDNA and modifying a target DNA sequence in an endogenous gene;

(l) one or more polynucleotides encoding a site-directed modifying polypeptide capable of interacting with a gDNA and modifying a target DNA sequence in an endogenous gene;

(m) one or more gRNAs capable of binding to a target mRNA sequence encoded by an endogenous gene;

(n) one or more polynucleotides encoding one or more gRNAs capable of binding to a target mRNA sequence encoded by an endogenous gene;

(o) one or more site-directed modifying polypeptides capable of interacting with a gRNA and modifying a target mRNA sequence encoded by an endogenous gene;

(p) one or more polynucleotides encoding a site-directed modifying polypeptide capable of interacting with a gRNA and modifying a target mRNA sequence encoded by an endogenous gene; or

(q) any combination of the above.

In some embodiments, one or more polynucleotides encoding the gene-regulating system are inserted into the genome of the Treg. In some embodiments, one or more polynucleotides encoding the gene-regulating system are expressed episomally and are not inserted into the genome of the Treg.

In some embodiments, the modified Tregs described herein comprise reduced expression and/or function of one or more endogenous target genes and further comprise one or more exogenous transgenes inserted at one or more genomic loci (e.g., a genetic “knock-in”). In some embodiments, the one or more exogenous transgenes encode detectable tags, safety-switch systems, chimeric switch receptors, and/or engineered antigen-specific receptors.

In some embodiments, the modified Tregs described herein further comprise an exogenous transgene encoding a detectable tag. Examples of detectable tags include but are not limited to, FLAG tags, poly-histidine tags (e.g. 6×His), SNAP tags, Halo tags, cMyc tags, glutathione-S-transferase tags, avidin, enzymes, fluorescent proteins, luminescent proteins, chemiluminescent proteins, bioluminescent proteins, and phosphorescent proteins. In some embodiments the fluorescent protein is selected from the group consisting of blue/UV proteins (such as BFP, TagBFP, mTagBFP2, Azurite, EBFP2, mKalama1, Sirius, Sapphire, and T-Sapphire); cyan proteins (such as CFP, eCFP, Cerulean, SCFP3A, mTurquoise, mTurquoise2, monomeric Midoriishi-Cyan, TagCFP, and mTFP1); green proteins (such as: GFP, eGFP, meGFP (A208K mutation), Emerald, Superfolder GFP, Monomeric Azami Green, TagGFP2, mUKG, mWasabi, Clover, and mNeonGreen); yellow proteins (such as YFP, eYFP, Citrine, Venus, SYFP2, and TagYFP); orange proteins (such as Monomeric Kusabira-Orange, mKOx, mKO2, mOrange, and mOrange2); red proteins (such as RFP, mRaspberry, mCherry, mStrawberry, mTangerine, tdTomato, TagRFP, TagRFP-T, mApple, mRuby, and mRuby2); far-red proteins (such as mPlum, HcRed-Tandem, mKate2, mNeptune, and NirFP); near-infrared proteins (such as TagRFP657, IFP1.4, and iRFP); long stokes shift proteins (such as mKeima Red, LSS-mKate1, LSS-mKate2, and mBeRFP); photoactivatable proteins (such as PA-GFP, PAmCherryl, and PATagRFP); photoconvertible proteins (such as Kaede (green), Kaede (red), KikGR1 (green), KikGR1 (red), PS-CFP2, PS-CFP2, mEos2 (green), mEos2 (red), mEos3.2 (green), mEos3.2 (red), PSmOrange, and PSmOrange); and photoswitchable proteins (such as Dronpa). In some embodiments, the detectable tag can be selected from AmCyan, AsRed, DsRed2, DsRed Express, E2-Crimson, HcRed, ZsGreen, ZsYellow, mCherry, mStrawberry, mOrange, mBanana, mPlum, mRasberry, tdTomato, DsRed Monomer, and/or AcGFP, all of which are available from Clontech.

In some embodiments, the modified Tregs described herein further comprise an exogenous transgene encoding a safety-switch system. Safety-switch systems (also referred to in the art as suicide gene systems) comprise exogenous transgenes encoding for one or more proteins that enable the elimination of a modified Treg after the cell has been administered to a subject. Examples of safety-switch systems are known in the art. For example, safety-switch systems include genes encoding for proteins that convert non-toxic pro-drugs into toxic compounds such as the Herpes simplex thymidine kinase (Hsv-tk) and ganciclovir (GCV) system (Hsv-tk/GCV). Hsv-tk converts non-toxic GCV into a cytotoxic compound that leads to cellular apoptosis. As such, administration of GCV to a subject that has been treated with modified Tregs comprising a transgene encoding the Hsv-tk protein can selectively eliminate the modified Tregs while sparing endogenous Tregs. (See e.g., Bonini et al., Science, 1997, 276(5319):1719-1724; Ciceri et al., Blood, 2007, 109(11):1828-1836; Bondanza et al., Blood 2006, 107(5):1828-1836).

Additional safety-switch systems include genes encoding for cell-surface markers, enabling elimination of modified Tregs by administration of a monoclonal antibody specific for the cell-surface marker via ADCC. In some embodiments, the cell-surface marker is CD20 and the modified Tregs can be eliminated by administration of an anti-CD20 monoclonal antibody such as Rituximab (See e.g., Introna et al., Hum Gene Ther, 2000, 11(4):611-620; Serafini et al., Hum Gene Ther, 2004, 14, 63-76; van Meerten et al., Gene Ther, 2006, 13, 789-797). Similar systems using EGF-R and Cetuximab or Panitumumab are described in International PCT Publication No. WO 2018006880. Additional safety-switch systems include transgenes encoding pro-apoptotic molecules comprising one or more binding sites for a chemical inducer of dimerization (CID), enabling elimination of modified Tregs by administration of a CID which induces oligomerization of the pro-apoptotic molecules and activation of the apoptosis pathway. In some embodiments, the pro-apoptotic molecule is Fas (also known as CD95) (Thomis et al., Blood, 2001, 97(5), 1249-1257). In some embodiments, the pro-apoptotic molecule is caspase-9 (Straathof et al., Blood, 2005, 105(11), 4247-4254).

In some embodiments, the modified Tregs described herein further comprise an exogenous transgene encoding a chimeric switch receptor. Chimeric switch receptors are engineered cell-surface receptors comprising an extracellular domain from an endogenous cell-surface receptor and a heterologous intracellular signaling domain, such that ligand recognition by the extracellular domain results in activation of a different signaling cascade than that activated by the wild type form of the cell-surface receptor. In some embodiments, the chimeric switch receptor comprises the extracellular domain of an inhibitory cell-surface receptor fused to an intracellular domain that leads to the transmission of an activating signal rather than the inhibitory signal normally transduced by the inhibitory cell-surface receptor. In particular embodiments, extracellular domains derived from cell-surface receptors known to inhibit Treg activation can be fused to activating intracellular domains. Engagement of the corresponding ligand will then activate signaling cascades that increase, rather than inhibit, the activation of the immune effector cell.

In some embodiments, the modified Tregs described herein further comprise an engineered antigen-specific receptor recognizing a protein target expressed by a target cell, referred to herein as “modified receptor-engineered cells” or “modified RE-cells”. The term “engineered antigen receptor” refers to a non-naturally occurring antigen-specific receptor such as a chimeric antigen receptor (CAR) or a recombinant T cell receptor (TCR). In some embodiments, the engineered antigen receptor is a CAR comprising an extracellular antigen binding domain fused via hinge and transmembrane domains to a cytoplasmic domain comprising a signaling domain. In some embodiments, the CAR extracellular domain binds to an antigen expressed by a target cell in an MIIC-independent manner leading to activation and proliferation of the RE cell. In some embodiments, the extracellular domain of a CAR recognizes a tag fused to an antibody or antigen-binding fragment thereof. In such embodiments, the antigen-specificity of the CAR is dependent on the antigen-specificity of the labeled antibody, such that a single CAR construct can be used to target multiple different antigens by substituting one antibody for another (See e.g., U.S. Pat. Nos. 9,233,125 and 9,624,279; US Patent Application Publication Nos. 20150238631 and 20180104354). In some embodiments, the extracellular domain of a CAR may comprise an antigen binding fragment derived from an antibody. Antigen binding domains that are useful in the present disclosure include, for example, scFvs; antibodies; antigen binding regions of antibodies; variable regions of the heavy/light chains; and single chain antibodies.

In some embodiments, the intracellular signaling domain of a CAR may be derived from the TCR complex zeta chain (such as CD3 signaling domains), FcγRIII, FcRI, or the T-lymphocyte activation domain. In some embodiments, the intracellular signaling domain of a CAR further comprises a costimulatory domain, for example a 4-1BB, CD28, CD40, MyD88, or CD70 domain. In some embodiments, the intracellular signaling domain of a CAR comprises two costimulatory domains, for example any two of 4-1BB, CD28, CD40, MyD88, or CD70 domains. Exemplary CAR structures and intracellular signaling domains are known in the art (See e.g., WO 2009/091826; US 20130287748; WO 2015/142675; WO 2014/055657; and WO 2015/090229, incorporated herein by reference).

CARs specific for antigens relevant for autoimmune diseases (e.g., GVHD, colitis, and multiple sclerosis) are discussed, for example, in Zhang et al., Frontiers in Immunology 9:1-8 (2018); Int'l Publ. No. WO2017218850A1; and McDonald et al., JCI 2016; 126(4):1413-1424, each of which is incorporated by reference herein in its entirety.

In some embodiments, the engineered antigen receptor is an engineered TCR. Engineered TCRs comprise TCRα and/or TCRβ chains that have been isolated and cloned from T cell populations recognizing a particular target antigen. For example, TCRα and/or TCRβ genes (i.e., TRAC and TRBC) can be cloned from T cell populations isolated from individuals with particular diseases or T cell populations that have been isolated from humanized mice immunized with cell types. Engineered TCRs recognize antigen through the same mechanisms as their endogenous counterparts (e.g., by recognition of their cognate antigen presented in the context of major histocompatibility complex (MHC) proteins expressed on the surface of a target cell). This antigen engagement stimulates endogenous signal transduction pathways leading to activation and proliferation of the TCR-engineered cells.

### A. Immunosuppressive Functions

In some embodiments, the modified Tregs described herein demonstrate an increase in one or more immunosuppressive functions, including the generation, maintenance, and/or enhancement of an immunosuppressive function. In some embodiments, the modified Tregs described herein demonstrate one or more of the following characteristics compared to an unmodified Treg: increased proliferation, increased or prolonged cell viability, improved stability, improved immunosuppressive function, or increased production of immunosuppressive immune factors (e.g., anti-inflammatory cytokines).

In some embodiments, the modified Tregs described herein demonstrate an increase in cell proliferation compared to an unmodified Treg. In these embodiments, the result is an increase in the number of modified Tregs present compared to unmodified Tregs after a given period of time. For example, in some embodiments, modified Tregs demonstrate increased rates of proliferation compared to unmodified Tregs, wherein the modified Tregs divide at a more rapid rate than unmodified Tregs. In some embodiments, the modified Tregs demonstrate a 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 25, 30, 35, 40, 45, 50, 60, 70, 80, 90, 100, or more fold increase in the rate of proliferation compared to an unmodified Treg. In some embodiments, modified Tregs demonstrate prolonged periods of proliferation compared to unmodified Tregs, wherein the modified Tregs and unmodified Tregs divide at similar rates, but wherein the modified Tregs maintain the proliferative state for a longer period of time. In some embodiments, the modified Tregs maintain a proliferative state for 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 25, 30, 35, 40, 45, 50, 60, 70, 80, 90, 100, or more times longer than an unmodified immune cell.

In some embodiments, the modified Tregs described herein demonstrate increased or prolonged cell viability compared to an unmodified Treg. In such embodiments, the result is an increase in the number of modified Tregs or present compared to unmodified Tregs after a given period of time. For example, in some embodiments, modified Tregs described herein remain viable and persist for 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 25, 30, 35, 40, 45, 50, 60, 70, 80, 90, 100, or more times longer than an unmodified immune cell.

In some embodiments, the modified Tregs described herein demonstrate increased resistance to Treg exhaustion compared to an unmodified Treg. In some embodiments, a 1.1, 1.2, 1.3, 1.4, 1.5, 1.6, 1.7, 1.8, 1.9, 2.0, 2.5, 3.0, 3.5, 4.0, 4.5, 5, 6, 7, 8, 9, 10, 15, 20, 30, 35, 40, 45, 50, 60, 70, 80, 90, 100 or more fold increase in cytokine production from the modified immune effector cells compared to the cytokine production from the control population of immune cells is indicative of an increased resistance to T cell exhaustion. In some embodiments, resistance to T cell exhaustion is demonstrated by increased proliferation of the modified immune effector cells compared to the proliferation observed from the control population of immune cells. In some embodiments, a 1.1, 1.2, 1.3, 1.4, 1.5, 1.6, 1.7, 1.8, 1.9, 2.0, 2.5, 3.0, 3.5, 4.0, 4.5, 5, 6, 7, 8, 9, 10, 15, 20, 30, 35, 40, 45, 50, 60, 70, 80, 90, 100 or more fold increase in proliferation of the modified immune effector cells compared to the proliferation of the control population of immune cells is indicative of an increased resistance to T cell exhaustion.

In some embodiments, exhaustion of the modified Tregs compared to control populations of immune cells is measured during the in vitro or ex vivo manufacturing process.

In some embodiments, the modified Tregs described herein demonstrate increased expression or production of anti-inflammatory immune factors compared to an unmodified Treg. Examples of anti-inflammatory or immunosuppressive immune factors include anti-inflammatory or immunosuppressive cytokines such as IL-10. In embodiments, the modified Tregs described herein demonstrate an improved stability. In embodiments, stability can be assessed, e.g., by measuring methylation of Foxp3 TSDR. In embodiments, the modified Tregs described herein demonstrate an improved immunosuppressive function. In some embodiments, the modified Tregs described herein have no impact on pro-inflammatory cytokines including IL-17A and IFNγ.

In some embodiments, the modified Tregs described herein demonstrate increased expression of Foxp3 and/or Helios compared to an unmodified Treg. In some embodiments, the modified Tregs described herein demonstrate increased coexpression of Foxp3 and Helios compared to an unmodified Treg.

Assays for measuring immunosuppressive function are known in the art. Cell-surface receptor expression can be determined by flow cytometry, immunohistochemistry, immunofluorescence, Western blot, and/or qPCR. Cytokine and chemokine expression and production can be measured by flow cytometry, immunohistochemistry, immunofluorescence, Western blot, ELISA, and/or qPCR. Responsiveness or sensitivity to extracellular stimuli (e.g., cytokines, inhibitory ligands, or antigen) can be measured by assaying cellular proliferation and/or activation of downstream signaling pathways (e.g., phosphorylation of downstream signaling intermediates) in response to the stimuli.

### B. Regulation of Endogenous Pathways and Genes

In some embodiments, the modified Tregs described herein demonstrate a reduced expression or function of one or more endogenous target genes. In some embodiments, the one or more endogenous target genes are present in pathways related to increased immunosuppressive function. In such embodiments, the reduced expression or function of the one or more endogenous target genes enhances one or more immunosuppressive functions of the immune cell.

Exemplary pathways suitable for regulation by the methods described herein include, for example, Treg proliferation, Treg viability, Treg stability, and/or Treg immunosuppressive activity pathways. In some embodiments, the expression of an endogenous target gene in a particular pathway is reduced in the modified Tregs. In some embodiments, the expression of a plurality (e.g., two or more) of endogenous target genes in a particular pathway are reduced in the modified Tregs. For example, the expression of 2, 3, 4, 5, 6, 7, 8, 9, 10, or more endogenous target genes in a particular pathway may be reduced. In some embodiments, the expression of an endogenous target gene in one pathway and the expression of an endogenous target genes in another pathway is reduced in the modified Tregs. In some embodiments, the expression of a plurality of endogenous target genes in one pathway and the expression of a plurality of endogenous target genes in another pathway are reduced in the modified Tregs. For example, the expression of 2, 3, 4, 5, 6, 7, 8, 9, 10, or more endogenous target genes in one pathway may be reduced and the expression of 2, 3, 4, 5, 6, 7, 8, 9, 10, or more endogenous target genes in another particular pathway may be reduced.

In some embodiments, the expression of a plurality of endogenous target genes in a plurality of pathways is reduced. For example, one endogenous gene from each of a plurality of pathways (e.g., 2, 3, 4, 5, 6, 7, 8, 9, 10, or more pathways) may be reduced. In additional aspects, a plurality of endogenous genes (e.g., 2, 3, 4, 5, 6, 7, 8, 9, 10, or more genes) from each of a plurality of pathways (e.g., 2, 3, 4, 5, 6, 7, 8, 9, 10, or more pathways) may be reduced.

Exemplary endogenous target genes are shown below in Table 1.

In some embodiments, expression of TNFRSF4 is reduced. TNFRSF4 is also known as “tumor necrosis factor superfamily member 4,” “ACT35 antigen,” “TNFRSF4L receptor,” “CD134,” “OX40,” and “TAX transcriptionally-activated glycoprotein 1 receptor.” TNFRSF4 is a receptor for TNFSF4 (also known as OX40L and GP34.) A soluble isoform of human TNFRSF4 has also been reported (Taylor L et al., (2001) J Immunol Methods 255: 67-72).

In some embodiments, expression of PRDM1 is reduced. PRDM1 is also known as “PR domain zinc finger protein 1”, “BLIMP1,” “PRDI-BF1,” and “beta-interferon gene positive regulatory domain I-binding factor.” PRDM1 is a transcription factor.

In some embodiments, the modified effector cells comprise reduced expression and/or function of one or more of TNFRSF4, PRDM1, REEP3, MRPL32, FSCN3, KLC3, C4BPA, LZTS1, CDK16, or ADNP. In some embodiments, the modified Tregs comprise reduced expression and/or function of a gene selected from Table 1. In some embodiments, the modified Tregs comprise reduced expression and/or function of at least two genes selected from Table 1 (e.g., at least two genes selected from TNFRSF4, PRDM1, REEP3, MRPL32, FSCN3, KLC3, C4BPA, LZTS1, CDK16, and ADNP). While exemplary methods for modifying the expression of TNFRSF4, PRDM1, REEP3, MRPL32, FSCN3, KLC3, C4BPA, LZTS1, CDK16, and ADNP described herein, the expression of these endogenous target genes may also be modified by methods known in the art.

In some embodiments, the modified effector cells comprise reduced expression of TNFRSF4. In some embodiments, the modified effector cells comprise reduced expression of PRDM1.

In some embodiments, the modified effector cells comprise reduced expression of TNFRSF4 and one or more of PRDM1, REEP3, MRPL32, FSCN3, KLC3, C4BPA, LZTS1, CDK16, and ADNP. In some embodiments, the modified Tregs comprise reduced expression of a gene selected from Table 1 and reduced expression of TNFRSF4. In some embodiments, the modified effector cells comprise reduced expression of PRDM1 and one or more of TNFRSF4, REEP3, MRPL32, FSCN3, KLC3, C4BPA, LZTS1, CDK16, and ADNP. In some embodiments, the modified Tregs comprise reduced expression of a gene selected from Table 1 and reduced expression of PRDM1. In some embodiments, the modified Tregs comprise reduced expression of TNFRSF4 and reduced expression of two genes selected from Table 1. In some embodiments, the modified Tregs comprise reduced expression of PRDM1 and reduced expression of two genes selected from Table 1. In some embodiments, the modified Tregs comprise reduced expression of a plurality of genes selected from Table 1 and reduced expression of TNFRSF4. In some embodiments, the modified Tregs comprise reduced expression of a plurality of genes selected from Table 1 and reduced expression of PRDM1. In some embodiments, the modified Tregs comprise reduced expression of two genes selected from Table 1 and reduced expression of TNFRSF4. In some embodiments, the modified Tregs comprise reduced expression of two genes selected from Table 1 and reduced expression of PRDM1. In some embodiments, the modified Tregs may comprise reduced expression of three or more of PRDM1, REEP3, MRPL32, FSCN3, KLC3, C4BPA, LZTS1, CDK16, and ADNP and reduced expression of TNFRSF4. In some embodiments, the modified Tregs may comprise reduced expression of three or more of TNFRSF4, REEP3, MRPL32, FSCN3, KLC3, C4BPA, LZTS1, CDK16, and ADNP and reduced expression of PRDM1.

In some embodiments, the expression of TNFRSF4 is reduced by a gene-regulating system described herein. In some embodiments, the expression of PRDM1 is reduced by a gene-regulating system described herein.

TABLE 1
Exemplary Endogenous Genes

| Gene | | Human | Murine |
|---|---|---|---|
| Symbol | Gene Name | UniProt Ref. | UniProt Ref. |
| PRDM1 | PR domain zinc finger | O75626 | Q60636 |
| | protein 1 | | |
| TNFRSF4 | Tumor necrosis factor | P43489 | P47741 |
| | receptor superfamily, | | |
| | member 4 | | |
| REEP3 | Receptor Accessory | Q6NUK4 | Q99KK1 |
| | Protein 3 | | |
| MRPL32 | 39S ribosomal protein | Q9BYC8 | Q9DCI9 |
| | L32, mitochondrial | | |
| FSCN3 | Fascin-3 | Q9NQT6 | Q9QXW4 |
| KLC3 | Kinesin light chain 3 | Q6P597 | Q91W40 |
| C4BPA | Complement | P04003 | P08607 |
| | Component 4 Binding | | |
| | Protein Alpha | | |
| LZTS1 | Leucine zipper putative | Q9Y250 | P60853 |
| | tumor suppressor 1 | | |
| CDK16 | Cyclin Dependent | Q00536 | Q04735 |
| | Kinase 16 | | |
| ADNP | Activity Dependent | Q9H2P0 | Q9Z103 |
| | Neuroprotector | | |
| | Homeobox | | |

### III. Gene-Regulating Systems

Herein, the term “gene-regulating system” refers to a protein, nucleic acid, or combination thereof that is capable of modifying an endogenous target DNA sequence when introduced into a cell, thereby regulating the expression or function of the encoded gene product. Numerous gene editing systems suitable for use in the methods of the present disclosure are known in the art including, but not limited to, shRNAs, siRNAs, zinc-finger nuclease systems, TALEN systems, and CRISPR/Cas systems.

As used herein, “regulate,” when used in reference to the effect of a gene-regulating system on an endogenous target gene encompasses any change in the sequence of the endogenous target gene, any change in the epigenetic state of the endogenous target gene, and/or any change in the expression or function of the protein encoded by the endogenous target gene.

In some embodiments, the gene-regulating system may mediate a change in the sequence of the endogenous target gene, for example, by introducing one or more mutations into the endogenous target sequence, such as by insertion or deletion of one or more nucleic acids in the endogenous target sequence. Exemplary mechanisms that can mediate alterations of the endogenous target sequence include, but are not limited to, non-homologous end joining (NHEJ) (e.g., classical or alternative), microhomology-mediated end joining (MMEJ), homology-directed repair (e.g., endogenous donor template mediated), SDSA (synthesis dependent strand annealing), single strand annealing or single strand invasion.

In some embodiments, the gene-regulating system may mediate a change in the epigenetic state of the endogenous target sequence. For example, in some embodiments, the gene-regulating system may mediate covalent modifications of the endogenous target gene DNA (e.g., cytosine methylation and hydroxymethylation) or of associated histone proteins (e.g. lysine acetylation, lysine and arginine methylation, serine and threonine phosphorylation, and lysine ubiquitination and sumoylation).

In some embodiments, the gene-regulating system may mediate a change in the expression of the protein encoded by the endogenous target gene. In such embodiments, the gene-regulating system may regulate the expression of the encoded protein by modifications of the endogenous target DNA sequence, or by acting on the mRNA product encoded by the DNA sequence. In some embodiments, the gene-regulating system may result in the expression of a modified endogenous protein. In such embodiments, the modifications to the endogenous DNA sequence mediated by the gene-regulating system result in the expression of an endogenous protein demonstrating a reduced function as compared to the corresponding endogenous protein in an unmodified Treg. In such embodiments, the expression level of the modified endogenous protein may be increased, decreased or may be the same, or substantially similar to, the expression level of the corresponding endogenous protein in an unmodified immune cell.

### A. Nucleic Acid-Based Gene-Regulating Systems

As used herein, a nucleic acid-based gene-regulating system is a system comprising one or more nucleic acid molecules that is capable of regulating the expression of an endogenous target gene without the requirement for an exogenous protein. In some embodiments, the nucleic acid-based gene-regulating system comprises an RNA interference molecule or antisense RNA molecule that is complementary to a target nucleic acid sequence.

An “antisense RNA molecule” refers to an RNA molecule, regardless of length, that is complementary to an mRNA transcript. Antisense RNA molecules refer to single stranded RNA molecules that can be introduced to a cell, tissue, or subject and result in decreased expression of an endogenous target gene product through mechanisms that do not rely on endogenous gene silencing pathways, but rather rely on RNaseH-mediated degradation of the target mRNA transcript. In some embodiments, an antisense nucleic acid comprises a modified backbone, for example, phosphorothioate, phosphorodithioate, or others known in the art, or may comprise non-natural internucleoside linkages. In some embodiments, an antisense nucleic acid can comprise locked nucleic acids (LNA).

“RNA interference molecule” as used herein refers to an RNA polynucleotide that mediates the decreased the expression of an endogenous target gene product by degradation of a target mRNA through endogenous gene silencing pathways (e.g., Dicer and RNA-induced silencing complex (RISC)). Exemplary RNA interference agents include micro RNAs (also referred to herein as “miRNAs”), short hair-pin RNAs (shRNAs), small interfering RNAs (siRNAs), RNA aptamers, and morpholinos.

In some embodiments, the nucleic acid-based gene-regulating system comprises one or more miRNAs. miRNAs refers to naturally occurring, small non-coding RNA molecules of about 21-25 nucleotides in length. miRNAs are at least partially complementary to one or more target mRNA molecules. miRNAs can downregulate (e.g., decrease) expression of an endogenous target gene product through translational repression, cleavage of the mRNA, and/or deadenylation.

In some embodiments, the nucleic acid-based gene-regulating system comprises one or more shRNAs. shRNAs are single stranded RNA molecules of about 50-70 nucleotides in length that form stem-loop structures and result in degradation of complementary mRNA sequences. shRNAs can be cloned in plasmids or in non-replicating recombinant viral vectors to be introduced intracellularly and result in the integration of the shRNA-encoding sequence into the genome. As such, an shRNA can provide stable and consistent repression of endogenous target gene translation and expression.

In some embodiments, nucleic acid-based gene-regulating system comprises one or more siRNAs. siRNAs refer to double stranded RNA molecules typically about 21-23 nucleotides in length. The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA, resulting in specific gene silencing. Optimally, an siRNA is 18, 19, 20, 21, 22, 23 or 24 nucleotides in length and has a 2 base overhang at its 3′ end. siRNAs can be introduced to an individual cell and/or culture system and result in the degradation of target mRNA sequences. siRNAs and shRNAs are further described in Fire et al., Nature, 391:19, 1998 and U.S. Pat. Nos. 7,732,417; 8,202,846; and 8,383,599.

In some embodiments, the nucleic acid-based gene-regulating system comprises one or more morpholinos. “Morpholino” as used herein refers to a modified nucleic acid oligomer wherein standard nucleic acid bases are bound to morpholine rings and are linked through phosphorodiamidate linkages. Similar to siRNA and shRNA, morpholinos bind to complementary mRNA sequences. However, morpholinos function through steric-inhibition of mRNA translation and alteration of mRNA splicing rather than targeting complementary mRNA sequences for degradation.

In some embodiments, the nucleic acid-based gene-regulating system comprises a nucleic acid molecule (e.g., an siRNA, an shRNA, an RNA aptamer, or a morpholino) that binds to a target RNA sequence that is at least 90% identical to an RNA encoded by a DNA sequence of a target gene selected from those listed in Table 1. In some embodiments, the nucleic acid-based gene-regulating system comprises a nucleic acid molecule (e.g., an siRNA, an shRNA, an RNA aptamer, or a morpholino) that bind to a target RNA sequence that is at least 95%, 96%, 97%, 98%, or 99% identical to an RNA encoded by a DNA sequence of a target gene selected from those listed in Table 1. In some embodiments, the nucleic acid-based gene-regulating system comprises a nucleic acid molecule (e.g., an siRNA, an shRNA, an RNA aptamer, or a morpholino) bind to a target RNA sequence that is 100% identical to an RNA encoded by a DNA sequence of a target gene selected from those listed in Table 1.

In some embodiments, the nucleic acid-based gene-regulating system comprises an siRNA molecule or an shRNA molecule selected from those known in the art, such as the siRNA and shRNA constructs available from commercial suppliers such as Sigma Aldrich, Dharmacon, ThermoFisher, and the like.

In some embodiments, the gene-regulating system comprises two or more nucleic acid molecules (e.g., two or more siRNAs, two or more shRNAs, two or more RNA aptamers, or two or more morpholinos), wherein at least one of the nucleic acid molecules binds to a target RNA sequence that is at least 90% identical to an RNA sequence encoded by a DNA sequence of a target gene selected from Table 1. In some embodiments, the gene-regulating system comprises two or more nucleic acid molecules (e.g., two or more siRNAs, two or more shRNAs, two or more RNA aptamers, or two or more morpholinos), wherein at least one of the nucleic acid molecules binds to a target RNA sequence that is at least 95%, 96%, 97%, 98%, or 99% identical to an RNA sequence encoded by a DNA sequence of a target gene selected from Table 1. In some embodiments, the gene-regulating system comprises two or more nucleic acid molecules (e.g., two or more siRNAs, two or more shRNAs, two or more RNA aptamers, or two or more morpholinos), wherein at least one of the nucleic acid molecules binds to a target RNA sequence that is 100% identical to an RNA sequence encoded by a DNA sequence of a target gene selected from Table 1.

### B. Protein-Based Gene-Regulating Systems

In some embodiments, a protein-based gene-regulating system is a system comprising one or more proteins capable of regulating the expression of an endogenous target gene in a sequence specific manner without the requirement for a nucleic acid guide molecule. In some embodiments, the protein-based gene-regulating system comprises a protein comprising one or more zinc-finger binding domains and an enzymatic domain. In some embodiments, the protein-based gene-regulating system comprises a protein comprising a Transcription activator-like effector nuclease (TALEN) domain and an enzymatic domain. Such embodiments are referred to herein as “TALENs”.

### 1. Zinc Finger Systems

Zinc finger-based systems comprise a fusion protein comprising two protein domains: a zinc finger DNA binding domain and an enzymatic domain. A “zinc finger DNA binding domain”, “zinc finger protein”, or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence, directs the activity of the enzymatic domain to the vicinity of the sequence and, hence, induces modification of the endogenous target gene in the vicinity of the target sequence. A zinc finger domain can be engineered to bind to virtually any desired sequence. Accordingly, after identifying a target genetic locus containing a target DNA sequence at which cleavage or recombination is desired (e.g., a target locus in a target gene referenced in Table 1), one or more zinc finger binding domains can be engineered to bind to one or more target DNA sequences in the target genetic locus. Expression of a fusion protein comprising a zinc finger binding domain and an enzymatic domain in a cell, effects modification in the target genetic locus.

In some embodiments, a zinc finger binding domain comprises one or more zinc fingers. Miller et al. (1985) EMBO J. 4:1609-1614; Rhodes (1993) Scientific American Febuary: 56-65; U.S. Pat. No. 6,453,242. Typically, a single zinc finger domain is about 30 amino acids in length. An individual zinc finger binds to a three-nucleotide (i.e., triplet) sequence (or a four-nucleotide sequence which can overlap, by one nucleotide, with the four-nucleotide binding site of an adjacent zinc finger). Therefore the length of a sequence to which a zinc finger binding domain is engineered to bind (e.g., a target sequence) will determine the number of zinc fingers in an engineered zinc finger binding domain. For example, for ZFPs in which the finger motifs do not bind to overlapping subsites, a six-nucleotide target sequence is bound by a two-finger binding domain; a nine-nucleotide target sequence is bound by a three-finger binding domain, etc. Binding sites for individual zinc fingers (i.e., subsites) in a target site need not be contiguous, but can be separated by one or several nucleotides, depending on the length and nature of the amino acids sequences between the zinc fingers (i.e., the inter-finger linkers) in a multi-finger binding domain. In some embodiments, the DNA-binding domains of individual ZFNs comprise between three and six individual zinc finger repeats and can each recognize between 9 and 18 basepairs.

Zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection.

Selection of a target DNA sequence for binding by a zinc finger domain can be accomplished, for example, according to the methods disclosed in U.S. Pat. No. 6,453,242. It will be clear to those skilled in the art that simple visual inspection of a nucleotide sequence can also be used for selection of a target DNA sequence. Accordingly, any means for target DNA sequence selection can be used in the methods described herein. A target site generally has a length of at least 9 nucleotides and, accordingly, is bound by a zinc finger binding domain comprising at least three zinc fingers. However binding of, for example, a 4-finger binding domain to a 12-nucleotide target site, a 5-finger binding domain to a 15-nucleotide target site or a 6-finger binding domain to an 18-nucleotide target site, is also possible. As will be apparent, binding of larger binding domains (e.g., 7-, 8-, 9-finger and more) to longer target sites is also possible.

In some embodiments, the zinc finger binding domains bind to a target DNA sequence that is at least 90% identical to a target DNA sequence of a target gene selected from those listed in Table 1. In some embodiments, the zinc finger binding domains bind to a target DNA sequence that is at least 95%, 96%, 97%, 98%, or 99% identical to a target DNA sequence of a target gene selected from those listed in Table 1. In some embodiments, the zinc finger binding domains bind to a target DNA sequence that is 100% identical to a target DNA sequence of a target gene selected from those listed in Table 1. In some embodiments, the zinc finger system is selected from those known in the art, such as those available from commercial suppliers such as Sigma Aldrich.

In some embodiments, the gene-regulating system comprises two or more ZFP-fusion proteins each comprising a zinc finger binding domain, wherein at least one of the zinc finger binding domains binds to a target DNA sequence that is at least 90% identical to a target DNA sequence of a target gene selected from Table 1. In some embodiments, the gene-regulating system comprises two or more ZFP-fusion proteins each comprising a zinc finger binding domain, wherein at least one of the zinc finger binding domains binds to a target DNA sequence that is at least 95%, 96%, 97%, 98%, or 99% identical to a target DNA sequence of a target gene selected from Table 1. In some embodiments, the gene-regulating system comprises two or more ZFP-fusion proteins each comprising a zinc finger binding domain, wherein at least one of the zinc finger binding domains binds to a target DNA sequence that is 100% identical to a target DNA sequence of a target gene selected from Table 1.

The enzymatic domain portion of the zinc finger fusion proteins can be obtained from any endo- or exonuclease. Exemplary endonucleases from which an enzymatic domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNaseI; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains.

Exemplary restriction endonucleases (restriction enzymes) suitable for use as an enzymatic domain of the ZFPs described herein are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the enzymatic domain from at least one Type IIS restriction enzyme and one or more zinc finger binding domains.

An exemplary Type IIS restriction enzyme, whose cleavage domain is separable from the binding domain, is FokI. This particular enzyme is active as a dimer. Bitinaite et al. (1998) Proc. Natl. Acad. Sci. USA 95: 10,570-10,575. Thus, for targeted double-stranded DNA cleavage using zinc finger-FokI fusions, two fusion proteins, each comprising a FokI enzymatic domain, can be used to reconstitute a catalytically active cleavage domain. Alternatively, a single polypeptide molecule containing a zinc finger binding domain and two FokI enzymatic domains can also be used. Exemplary ZFPs comprising FokI enzymatic domains are described in U.S. Pat. No. 9,782,437.

### 2. TALEN Systems

TALEN-based systems comprise a protein comprising a TAL effector DNA binding domain and an enzymatic domain. They are made by fusing a TAL effector DNA-binding domain to a DNA cleavage domain (a nuclease which cuts DNA strands). The FokI restriction enzyme described above is an exemplary enzymatic domain suitable for use in TALEN-based gene-regulating systems.

TAL effectors are proteins that are secreted by Xanthomonas bacteria via their type III secretion system when they infect plants. The DNA binding domain contains a repeated, highly conserved, 33-34 amino acid sequence with divergent 12th and 13th amino acids. These two positions, referred to as the Repeat Variable Diresidue (RVD), are highly variable and strongly correlated with specific nucleotide recognition. Therefore, the TAL effector domains can be engineered to bind specific target DNA sequences by selecting a combination of repeat segments containing the appropriate RVDs. The nucleic acid specificity for RVD combinations is as follows: HD targets cytosine, NI targets adenenine, NG targets thymine, and NN targets guanine (though, in some embodiments, NN can also bind adenenine with lower specificity).

In some embodiments, the TAL effector domains bind to a target DNA sequence that is at least 90% identical to a target DNA sequence of a target gene selected from those listed in Table 1. In some embodiments, the TAL effector domains bind to a target DNA sequence that is at least 95%, 96%, 97%, 98%, or 99% identical to a target DNA sequence of a target gene selected those listed in Table 1. In some embodiments, the TAL effector domains bind to a target DNA sequence that is 100% identical to a target DNA sequence of a target gene selected from those listed in Table 1.

In some embodiments, the gene-regulating system comprises two or more TAL effector-fusion proteins each comprising a TAL effector domain, wherein at least one of the TAL effector domains binds to a target DNA sequence that is at least 90% identical to a target DNA sequence of a target gene selected from Table 1. In some embodiments, the gene-regulating system comprises two or more TAL effector-fusion proteins each comprising a TAL effector domain, wherein at least one of the TAL effector domains binds to a target DNA sequence that is at least 95%, 96%, 97%, 98%, or 99% identical to a target DNA sequence of a target gene selected from Table 1. In some embodiments, the gene-regulating system comprises two or more TAL effector-fusion proteins each comprising a TAL effector domain, wherein at least one of the TAL effector domains binds to a target DNA sequence that is 100% identical to a target DNA sequence of a target gene selected from Table 1.

Methods and compositions for assembling the TAL-effector repeats are known in the art. See e.g., Cermak et al, Nucleic Acids Research, 39:12, 2011, e82. Plasmids for constructions of the TAL-effector repeats are commercially available from Addgene.

### C. Combination Nucleic Acid/Protein-Based Gene-Regulating Systems

Combination gene-regulating systems comprise a site-directed modifying polypeptide and a nucleic acid guide molecule. Herein, a “site-directed modifying polypeptide” refers to a polypeptide that binds to a nucleic acid guide molecule, is targeted to a target nucleic acid sequence, (for example, an endogenous target DNA or RNA sequence) by the nucleic acid guide molecule to which it is bound, and modifies the target nucleic acid sequence (e.g., cleavage, mutation, or methylation of a target nucleic acid sequence).

A site-directed modifying polypeptide comprises two portions, a portion that binds the nucleic acid guide and an activity portion. In some embodiments, a site-directed modifying polypeptide comprises an activity portion that exhibits site-directed enzymatic activity (e.g., DNA methylation, DNA or RNA cleavage, histone acetylation, histone methylation, etc.), wherein the site of enzymatic activity is determined by the guide nucleic acid. In some cases, a site-directed modifying polypeptide comprises an activity portion that has enzymatic activity that modifies the endogenous target nucleic acid sequence (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). In other cases, a site-directed modifying polypeptide comprises an activity portion that has enzymatic activity that modifies a polypeptide (e.g., a histone) associated with the endogenous target nucleic acid sequence (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). In some embodiments, a site-directed modifying polypeptide comprises an activity portion that modulates transcription of a target DNA sequence (e.g., to increase or decrease transcription). In some embodiments, a site-directed modifying polypeptide comprises an activity portion that modulates expression or translation of a target RNA sequence (e.g., to increase or decrease transcription).

The nucleic acid guide comprises two portions: a first portion that is complementary to, and capable of binding with, an endogenous target nucleic sequence (referred to herein as a “nucleic acid-binding segment”), and a second portion that is capable of interacting with the site-directed modifying polypeptide (referred to herein as a “protein-binding segment”). In some embodiments, the nucleic acid-binding segment and protein-binding segment of a nucleic acid guide are comprised within a single polynucleotide molecule. In some embodiments, the nucleic acid-binding segment and protein-binding segment of a nucleic acid guide are each comprised within separate polynucleotide molecules, such that the nucleic acid guide comprises two polynucleotide molecules that associate with each other to form the functional guide.

The nucleic acid guide mediates the target specificity of the combined protein/nucleic acid gene-regulating systems by specifically hybridizing with a target nucleic acid sequence. In some embodiments, the target nucleic acid sequence is an RNA sequence, such as an RNA sequence comprised within an mRNA transcript of a target gene. In some embodiments, the target nucleic acid sequence is a DNA sequence comprised within the DNA sequence of a target gene. Reference herein to a target gene encompasses the full-length DNA sequence for that particular gene which comprises a plurality of target genetic loci (i.e., portions of a particular target gene sequence (e.g., an exon or an intron)). Within each target genetic loci are shorter stretches of DNA sequences referred to herein as “target DNA sequences” that can be modified by the gene-regulating systems described herein. Further, each target genetic loci comprises a “target modification site,” which refers to the precise location of the modification induced by the gene-regulating system (e.g., the location of an insertion, a deletion, or mutation, the location of a DNA break, or the location of an epigenetic modification).

The gene-regulating systems described herein may comprise a single nucleic acid guide, or may comprise a plurality of nucleic acid guides (e.g., 2, 3, 4, 5, 6, 7, 8, 9, 10, or more nucleic acid guides).

In some embodiments, the combined protein/nucleic acid gene-regulating systems comprise site-directed modifying polypeptides derived from Argonaute (Ago) proteins (e.g., T. thermophiles Ago or TtAgo). In such embodiments, the site-directed modifying polypeptide is a T. thermophiles Ago DNA endonuclease and the nucleic acid guide is a guide DNA (gDNA) (See, Swarts et al., Nature 507 (2014), 258-261). In some embodiments, the present disclosure provides a polynucleotide encoding a gDNA. In some embodiments, a gDNA-encoding nucleic acid is comprised in an expression vector, e.g., a recombinant expression vector. In some embodiments, the present disclosure provides a polynucleotide encoding a TtAgo site-directed modifying polypeptide or variant thereof. In some embodiments, the polynucleotide encoding a TtAgo site-directed modifying polypeptide is comprised in an expression vector, e.g., a recombinant expression vector.

In some embodiments, the gene editing systems described herein are CRISPR (Clustered Regularly Interspaced Short Palindromic Repeats)/Cas (CRISPR Associated) nuclease systems. In some embodiments, the CRISPR/Cas system is a Class 2 system. Class 2 CRISPR/Cas systems are divided into three types: Type II, Type V, and Type VI systems. In some embodiments, the CRISPR/Cas system is a Class 2 Type II system, utilizing the Cas9 protein. In such embodiments, the site-directed modifying polypeptide is a Cas9 DNA endonuclease (or variant thereof) and the nucleic acid guide molecule is a guide RNA (gRNA). In some embodiments, the CRISPR/Cas system is a Class 2 Type V system, utilizing the Cas12 proteins (e.g., Cas12a (also known as Cpf1), Cas12b (also known as C2c1), Cas12c (also known as C2c3), Cas12d (also known as CasY), and Cas12e (also known as CasX)). In such embodiments, the site-directed modifying polypeptide is a Cas12 DNA endonuclease (or variant thereof) and the nucleic acid guide molecule is a gRNA. In some embodiments, the CRISPR/Cas system is a Class 2 and Type VI system, utilizing the Cas13 proteins (e.g., Cas13a (also known as C2c2), Cas13b, and Cas13c). (See, Pyzocha et al., ACS Chemical Biology, 13(2), 347-356). In such embodiments, the site-directed modifying polypeptide is a Cas13 RNA riboendonuclease and the nucleic acid guide molecule is a gRNA.

A Cas polypeptide refers to a polypeptide that can interact with a gRNA molecule and, in concert with the gRNA molecule, home or localize to a target DNA or target RNA sequence. Cas polypeptides include naturally occurring Cas proteins and engineered, altered, or otherwise modified Cas proteins that differ by one or more amino acid residues from a naturally-occurring Cas sequence.

A guide RNA (gRNA) comprises two segments, a DNA-binding segment and a protein-binding segment. In some embodiments, the protein-binding segment of a gRNA is comprised in one RNA molecule and the DNA-binding segment is comprised in another separate RNA molecule. Such embodiments are referred to herein as “double-molecule gRNAs” or “two-molecule gRNA” or “dual gRNAs.” In some embodiments, the gRNA is a single RNA molecule and is referred to herein as a “single-guide RNA” or an “sgRNA.” The term “guide RNA” or “gRNA” is inclusive, referring both to two-molecule guide RNAs and sgRNAs.

The protein-binding segment of a gRNA comprises, in part, two complementary stretches of nucleotides that hybridize to one another to form a double stranded RNA duplex (dsRNA duplex), which facilitates binding to the Cas protein. The nucleic acid-binding segment (or “nucleic acid-binding sequence”) of a gRNA comprises a nucleotide sequence that is complementary to and capable of binding to a specific target nucleic acid sequence. The protein-binding segment of the gRNA interacts with a Cas polypeptide and the interaction of the gRNA molecule and site-directed modifying polypeptide results in Cas binding to the endogenous nucleic acid sequence and produces one or more modifications within or around the target nucleic acid sequence. The precise location of the target modification site is determined by both (i) base-pairing complementarity between the gRNA and the target nucleic acid sequence; and (ii) the location of a short motif, referred to as the protospacer adjacent motif (PAM), in the target DNA sequence (referred to as a protospacer flanking sequence (PFS) in target RNA sequences). The PAM/PFS sequence is required for Cas binding to the target nucleic acid sequence. A variety of PAM/PFS sequences are known in the art and are suitable for use with a particular Cas endonuclease (e.g., a Cas9 endonuclease) (See e.g., Nat Methods. 2013 November; 10(11): 1116-1121 and Sci Rep. 2014; 4: 5405). In some embodiments, the PAM sequence is located within 50 base pairs of the target modification site in a target DNA sequence. In some embodiments, the PAM sequence is located within 10 base pairs of the target modification site in a target DNA sequence. The DNA sequences that can be targeted by this method are limited only by the relative distance of the PAM sequence to the target modification site and the presence of a unique 20 base pair sequence to mediate sequence-specific, gRNA-mediated Cas binding. In some embodiments, the PFS sequence is located at the 3′ end of the target RNA sequence. In some embodiments, the target modification site is located at the 5′ terminus of the target locus. In some embodiments, the target modification site is located at the 3′ end of the target locus. In some embodiments, the target modification site is located within an intron or an exon of the target locus.

In some embodiments, the present disclosure provides a polynucleotide encoding a gRNA. In some embodiments, a gRNA-encoding nucleic acid is comprised in an expression vector, e.g., a recombinant expression vector. In some embodiments, the present disclosure provides a polynucleotide encoding a site-directed modifying polypeptide. In some embodiments, the polynucleotide encoding a site-directed modifying polypeptide is comprised in an expression vector, e.g., a recombinant expression vector.

### 1. Cas Proteins

In some embodiments, the site-directed modifying polypeptide is a Cas protein. Any Cas protein, including those provided herein, can be used. Cas molecules of a variety of species can be used in the methods and compositions described herein, including Cas molecules derived from S. pyogenes, S. aureus, N. meningitidis, S. thermophiles, Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterospoxus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputomm, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae.

In some embodiments, the Cas protein is a naturally-occurring Cas protein. In some embodiments, the Cas endonuclease is selected from the group consisting of C2C1, C2C3, Cpf1 (also referred to as Cas12a), Cas12b, Cas12c, Cas12d, Cas12e, Cas13a, Cas13b, Cas13c, Cas13d, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some embodiments, the Cas protein is an endoribonuclease such as a Cas13 protein. In some embodiments, the Cas13 protein is a Cas13a (Abudayyeh et al., Nature 550 (2017), 280-284), Cas13b (Cox et al., Science (2017) 358:6336, 1019-1027), Cas13c (Cox et al., Science (2017) 358:6336, 1019-1027), or Cas13d (Zhang et al., Cell 175 (2018), 212-223) protein.

In some embodiments, the Cas9 protein is any Cas9 protein, including any of the Cas9 proteins specifically provided herein. In some embodiments, the Cas protein is a wild-type or naturally occurring Cas9 protein or a Cas9 ortholog. Wild-type Cas9 is a multi-domain enzyme that uses an HNH nuclease domain to cleave the target strand of DNA and a RuvC-like domain to cleave the non-target strand. Binding of WT Cas9 to DNA based on gRNA specificity results in double-stranded DNA breaks that can be repaired by non-homologous end joining (NHEJ) or homology-directed repair (HDR). Exemplary naturally occurring Cas9 molecules are described in Chylinski et al., RNA Biology 2013 10:5, 727-737 and additional Cas9 orthologs are described in International PCT Publication No. WO 2015/071474. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

In some embodiments, the naturally occurring Cas9 polypeptide is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the Cas9 protein comprises an amino acid sequence having at least 60%, 65%, 70%, 75%, 80%, 85%, 90%, 95%, 96%, 97%, 98%, 99%, or 100% sequence identity to a Cas9 amino acid sequence described in Chylinski et al., RNA Biology 2013 10:5, 727-737; Hou et al., PNAS Early Edition 2013, 1-6).

In some embodiments, the Cas polypeptide comprises one or more of the following activities:

(a) a nickase activity, i.e., the ability to cleave a single strand, e.g., the non-complementary strand or the complementary strand, of a nucleic acid molecule;

(b) a double stranded nuclease activity, i.e., the ability to cleave both strands of a double stranded nucleic acid and create a double stranded break, which in an embodiment is the presence of two nickase activities;

(c) an endonuclease activity;

(d) an exonuclease activity; and/or

(e) a helicase activity, i.e., the ability to unwind the helical structure of a double stranded nucleic acid.

In some embodiments, the Cas polypeptide is fused to heterologous proteins that recruit DNA-damage signaling proteins, exonucleases, or phosphatases to further increase the likelihood or the rate of repair of the target sequence by one repair mechanism or another. In some embodiments, a WT Cas polypeptide is co-expressed with a nucleic acid repair template to facilitate the incorporation of an exogenous nucleic acid sequence by homology-directed repair.

In some embodiments, different Cas proteins (i.e., Cas9 proteins from various species) may be advantageous to use in the various provided methods in order to capitalize on various enzymatic characteristics of the different Cas proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.).

In some embodiments, the Cas protein is a Cas9 protein derived from S. pyogenes and recognizes the PAM sequence motif NGG, NAG, NGA (Mali et al, Science 2013; 339(6121): 823-826). In some embodiments, the Cas protein is a Cas9 protein derived from S. thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the Cas protein is a Cas9 protein derived from S. mutans and recognizes the PAM sequence motif NGG and/or NAAR (R=A or G) (See, e.g., Deveau et al, J BACTERIOL 2008; 190(4): 1390-1400). In some embodiments, the Cas protein is a Cas9 protein derived from S. aureus and recognizes the PAM sequence motif NNGRR (R=A or G). In some embodiments, the Cas protein is a Cas9 protein derived from S. aureus and recognizes the PAM sequence motif N GRRT (R=A or G). In some embodiments, the Cas protein is a Cas9 protein derived from S. aureus and recognizes the PAM sequence motif N GRRV (R=A or G). In some embodiments, the Cas protein is a Cas9 protein derived from N. meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the Cas protein is a Cas13a protein derived from Leptotrichia shahii and recognizes the PFS sequence motif of a single 3′ A, U, or C.

In some embodiments, a polynucleotide encoding a Cas protein is provided. In some embodiments, the polynucleotide encodes a Cas protein that is at least 90% identical to a Cas protein described in International PCT Publication No. WO 2015/071474 or Chylinski et al., RNA Biology 2013 10:5, 727-737. In some embodiments, the polynucleotide encodes a Cas protein that is at least 95%, 96%, 97%, 98%, or 99% identical to a Cas protein described in International PCT Publication No. WO 2015/071474 or Chylinski et al., RNA Biology 2013 10:5, 727-737. In some embodiments, the polynucleotide encodes a Cas protein that is 100% identical to a Cas protein described in International PCT Publication No. WO 2015/071474 or Chylinski et al., RNA Biology 2013 10:5, 727-737.

### 2. Cas Mutants

In some embodiments, the Cas polypeptides are engineered to alter one or more properties of the Cas polypeptide. For example, in some embodiments, the Cas polypeptide comprises altered enzymatic properties, e.g., altered nuclease activity, (as compared with a naturally occurring or other reference Cas molecule) or altered helicase activity. In some embodiments, an engineered Cas polypeptide can have an alteration that alters its size, e.g., a deletion of amino acid sequence that reduces its size without significant effect on another property of the Cas polypeptide. In some embodiments, an engineered Cas polypeptide comprises an alteration that affects PAM recognition. For example, an engineered Cas polypeptide can be altered to recognize a PAM sequence other than the PAM sequence recognized by the corresponding wild-type Cas protein.

Cas polypeptides with desired properties can be made in a number of ways, including alteration of a naturally occurring Cas polypeptide or parental Cas polypeptide, to provide a mutant or altered Cas polypeptide having a desired property. For example, one or more mutations can be introduced into the sequence of a parental Cas polypeptide (e.g., a naturally occurring or engineered Cas polypeptide). Such mutations and differences may comprise substitutions (e.g., conservative substitutions or substitutions of non-essential amino acids); insertions; or deletions. In some embodiments, a mutant Cas polypeptide comprises one or more mutations (e.g., at least 1, 2, 3, 4, 5, 10, 15, 20, 30, 40 or 50 mutations) relative to a parental Cas polypeptide.

In an embodiment, a mutant Cas polypeptide comprises a cleavage property that differs from a naturally occurring Cas polypeptide. In some embodiments, the Cas is a deactivated Cas (dCas) mutant. In such embodiments, the Cas polypeptide does not comprise any intrinsic enzymatic activity and is unable to mediate target nucleic acid cleavage. In such embodiments, the dCas may be fused with a heterologous protein that is capable of modifying the target nucleic acid in a non-cleavage based manner. For example, in some embodiments, a dCas protein is fused to transcription activator or transcription repressor domains (e.g., the Kruppel associated box (KRAB or SKD); the Mad mSIN3 interaction domain (SID or SID4X); the ERF repressor domain (ERD); the MAX-interacting protein 1 (MXI1); methyl-CpG binding protein 2 (MECP2); etc.). In some such cases, the dCas fusion protein is targeted by the gRNA to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target DNA or modifies a polypeptide associated with the target DNA). In some cases, the changes are transient (e.g., transcription repression or activation). In some cases, the changes are inheritable (e.g., when epigenetic modifications are made to the target DNA or to proteins associated with the target DNA, e.g., nucleosomal histones).

In some embodiments, the dCas is a dCas13 mutant (Konermann et al., Cell 173 (2018), 665-676). These dCas13 mutants can then be fused to enzymes that modify RNA, including adenosine deaminases (e.g., ADAR1 and ADAR2). Adenosine deaminases convert adenine to inosine, which the translational machinery treats like guanine, thereby creating a functional A→G change in the RNA sequence. In some embodiments, the dCas is a dCas9 mutant.

In some embodiments, the mutant Cas9 is a Cas9 nickase mutant. Cas9 nickase mutants comprise only one catalytically active domain (either the HNH domain or the RuvC domain). The Cas9 nickase mutants retain DNA binding based on gRNA specificity, but are capable of cutting only one strand of DNA resulting in a single-strand break (e.g. a “nick”). In some embodiments, two complementary Cas9 nickase mutants (e.g., one Cas9 nickase mutant with an inactivated RuvC domain, and one Cas9 nickase mutant with an inactivated HNH domain) are expressed in the same cell with two gRNAs corresponding to two respective target sequences; one target sequence on the sense DNA strand, and one on the antisense DNA strand. This dual-nickase system results in staggered double stranded breaks and can increase target specificity, as it is unlikely that two off-target nicks will be generated close enough to generate a double stranded break. In some embodiments, a Cas9 nickase mutant is co-expressed with a nucleic acid repair template to facilitate the incorporation of an exogenous nucleic acid sequence by homology-directed repair.

In some embodiments, the Cas polypeptides described herein can be engineered to alter the PAM/PFS specificity of the Cas polypeptide. In some embodiments, a mutant Cas polypeptide has a PAM/PFS specificity that is different from the PAM/PFS specificity of the parental Cas polypeptide. For example, a naturally occurring Cas protein can be modified to alter the PAM/PFS sequence that the mutant Cas polypeptide recognizes to decrease off target sites, improve specificity, or eliminate a PAM/PFS recognition requirement. In some embodiments, a Cas protein can be modified to increase the length of the PAM/PFS recognition sequence. In some embodiments, the length of the PAM recognition sequence is at least 4, 5, 6, 7, 8, 9, 10 or 15 amino acids in length. Cas polypeptides that recognize different PAM/PFS sequences and/or have reduced off-target activity can be generated using directed evolution. Exemplary methods and systems that can be used for directed evolution of Cas polypeptides are described, e.g., in Esvelt et al. Nature 2011, 472(7344): 499-503.

Exemplary Cas mutants are described in International PCT Publication No. WO 2015/161276 and Konermann et al., Cell 173 (2018), 665-676, which are incorporated herein by reference in their entireties.

3. gRNAs

The present disclosure provides guide RNAs (gRNAs) that direct a site-directed modifying polypeptide to a specific target nucleic acid sequence. A gRNA comprises a nucleic acid-targeting segment and protein-binding segment. The nucleic acid-targeting segment of a gRNA comprises a nucleotide sequence that is complementary to a sequence in the target nucleic acid sequence. As such, the nucleic acid-targeting segment of a gRNA interacts with a target nucleic acid in a sequence-specific manner via hybridization (i.e., base pairing), and the nucleotide sequence of the nucleic acid-targeting segment determines the location within the target nucleic acid that the gRNA will bind. The nucleic acid-targeting segment of a gRNA can be modified (e.g., by genetic engineering) to hybridize to any desired sequence within a target nucleic acid sequence.

The protein-binding segment of a guide RNA interacts with a site-directed modifying polypeptide (e.g. a Cas protein) to form a complex. The guide RNA guides the bound polypeptide to a specific nucleotide sequence within target nucleic acid via the above-described nucleic acid-targeting segment. The protein-binding segment of a guide RNA comprises two stretches of nucleotides that are complementary to one another and which form a double stranded RNA duplex.

In some embodiments, a gRNA comprises two separate RNA molecules. In such embodiments, each of the two RNA molecules comprises a stretch of nucleotides that are complementary to one another such that the complementary nucleotides of the two RNA molecules hybridize to form the double-stranded RNA duplex of the protein-binding segment. In some embodiments, a gRNA comprises a single RNA molecule (sgRNA).

The specificity of a gRNA for a target loci is mediated by the sequence of the nucleic acid-binding segment, which comprises about 20 nucleotides that are complementary to a target nucleic acid sequence within the target locus. In some embodiments, the corresponding target nucleic acid sequence is approximately 20 nucleotides in length. In some embodiments, the nucleic acid-binding segments of the gRNA sequences of the present disclosure are at least 90% complementary to a target nucleic acid sequence within a target locus. In some embodiments, the nucleic acid-binding segments of the gRNA sequences of the present disclosure are at least 95%, 96%, 97%, 98%, or 99% complementary to a target nucleic acid sequence within a target locus. In some embodiments, the nucleic acid-binding segments of the gRNA sequences of the present disclosure are 100% complementary to a target nucleic acid sequence within a target locus.

In some embodiments, the target nucleic acid sequence is an RNA target sequence. In some embodiments, the target nucleic acid sequence is a DNA target sequence. In some embodiments, the nucleic acid-binding segments of the gRNA sequences bind to a target DNA sequence that is at least 90% identical to a target DNA sequence of a target gene selected from those listed in Table 1. In some embodiments, the nucleic acid-binding segments of the gRNA sequences bind to a target DNA sequence that is at least 95%, 96%, 97%, 98%, or 99% identical to a target DNA sequence of a target gene selected from those listed in Table 1. In some embodiments, the nucleic acid-binding segments of the gRNA sequences bind to a target DNA sequence that is 100% identical to a target DNA sequence of a target gene selected from those listed in Table 1.

In some embodiments, the gene-regulating system comprises two or more gRNA molecules each comprising a DNA-binding segment, wherein at least one of the nucleic acid-binding segments binds to a target DNA sequence that is at least 90% identical to a target DNA sequence of a target gene selected from Table 1. In some embodiments, the gene-regulating system comprises two or more gRNA molecules each comprising a nucleic acid-binding segment, wherein at least one of the nucleic acid-binding segments binds to a target DNA sequence that is at least 95%, 96%, 97%, 98%, or 99% identical to a target DNA sequence of a target gene selected from Table 1. In some embodiments, the gene-regulating system comprises two or more gRNA molecules each comprising a nucleic acid-binding segment, wherein at least one of the nucleic acid-binding segments binds to a target DNA sequence that is 100% to a target DNA sequence of a target gene selected from Table 1.

In some embodiments, the nucleic acid-binding segments of the gRNA sequences described herein are designed to minimize off-target binding using algorithms known in the art (e.g., Cas-OFF finder) to identify target sequences that are unique to a particular target locus or target gene.

In some embodiments, the gRNAs described herein can comprise one or more modified nucleosides or nucleotides which introduce stability toward nucleases. In such embodiments, these modified gRNAs may elicit a reduced innate immune response as compared to a non-modified gRNA. The term “innate immune response” includes a cellular response to exogenous nucleic acids, including single stranded nucleic acids, generally of viral or bacterial origin, which involves the induction of cytokine expression and release, particularly the interferons, and cell death.

In some embodiments, the gRNAs described herein are modified at or near the 5′ end (e.g., within 1-10, 1-5, or 1-2 nucleotides of their 5′ end). In some embodiments, the 5′ end of a gRNA is modified by the inclusion of a eukaryotic mRNA cap structure or cap analog (e.g., a G(5′)ppp(5′)G cap analog, a m7G(5′)ppp(5′)G cap analog, or a 3′-0-Me-m7G(5′)ppp(5′)G anti reverse cap analog (ARCA)). In some embodiments, an in vitro transcribed gRNA is modified by treatment with a phosphatase (e.g., calf intestinal alkaline phosphatase) to remove the 5′ triphosphate group. In some embodiments, a gRNA comprises a modification at or near its 3′ end (e.g., within 1-10, 1-5, or 1-2 nucleotides of its 3′ end). For example, in some embodiments, the 3′ end of a gRNA is modified by the addition of one or more (e.g., 25-200) adenine (A) residues.

In some embodiments, modified nucleosides and modified nucleotides can be present in a gRNA, but also may be present in other gene-regulating systems, e.g., mRNA, RNAi, or siRNA-based systems. In some embodiments, modified nucleosides and nucleotides can include one or more of:

(a) alteration, e.g., replacement, of one or both of the non-linking phosphate oxygens and/or of one or more of the linking phosphate oxygens in the phosphodiester backbone linkage;

(b) alteration, e.g., replacement, of a constituent of the ribose sugar, e.g., of the 2′ hydroxyl on the ribose sugar;

(c) wholesale replacement of the phosphate moiety with “dephospho” linkers;

(d) modification or replacement of a naturally occurring nucleobase;

(e) replacement or modification of the ribose-phosphate backbone;

(f) modification of the 3′ end or 5′ end of the oligonucleotide, e.g., removal, modification or replacement of a terminal phosphate group or conjugation of a moiety; and

(g) modification of the sugar.

In some embodiments, the modifications listed above can be combined to provide modified nucleosides and nucleotides that can have two, three, four, or more modifications. For example, in some embodiments, a modified nucleoside or nucleotide can have a modified sugar and a modified nucleobase. In some embodiments, every base of a gRNA is modified. In some embodiments, each of the phosphate groups of a gRNA molecule are replaced with phosphorothioate groups.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool.

### IV. Methods of Producing Modified Regulatory T Cell

In some embodiments, the present disclosure provides methods for producing modified Tregs. In some embodiments, the methods comprise introducing a gene-regulating system into a population of Tregs wherein the gene-regulating system is capable of reducing expression and/or function of one or more endogenous target genes.

The components of the gene-regulating systems described herein, e.g., a nucleic acid-, protein-, or nucleic acid/protein-based system can be introduced into target cells in a variety of forms using a variety of delivery methods and formulations. In some embodiments, a polynucleotide encoding one or more components of the system is delivered by a recombinant vector (e.g., a viral vector or plasmid). In some embodiments, where the system comprises more than a single component, a vector may comprise a plurality of polynucleotides, each encoding a component of the system. In some embodiments, where the system comprises more than a single component, a plurality of vectors may be used, wherein each vector comprises a polynucleotide encoding a particular component of the system. In some embodiments, a vector may also comprise a sequence encoding a signal peptide (e.g., for nuclear localization, nucleolar localization, mitochondrial localization), fused to the polynucleotide encoding the one or more components of the system. For example, a vector may comprise a nuclear localization sequence (e.g., from SV40) fused to the polynucleotide encoding the one or more components of the system. In some embodiments, the introduction of the gene-regulating system to the cell occurs in vitro. In some embodiments, the introduction of the gene-regulating system to the cell occurs in vivo. In some embodiments, the introduction of the gene-regulating system to the cell occurs ex vivo.

In some embodiments, the recombinant vector comprising a polynucleotide encoding one or more components of a gene-regulating system described herein is a viral vector. Suitable viral vectors include, but are not limited to, viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., U.S. Pat. No. 7,078,387; Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

In some embodiments, the recombinant vector comprising a polynucleotide encoding one or more components of a gene-regulating system described herein is a plasmid. Numerous suitable plasmid expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other plasmid vector may be used so long as it is compatible with the host cell. Depending on the cell type and gene-regulating system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a polynucleotide sequence encoding one or more components of a gene-regulating system described herein is operably linked to a control element, e.g., a transcriptional control element, such as a promoter. The transcriptional control element may be functional in either a eukaryotic cell (e.g., a mammalian cell) or a prokaryotic cell (e.g., bacterial or archaeal cell). In some embodiments, a polynucleotide sequence encoding one or more components of a gene-regulating system described herein is operably linked to multiple control elements that allow expression of the polynucleotide in both prokaryotic and eukaryotic cells. Depending on the cell type and gene-regulating system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-1. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, green fluorescent protein, etc.) that are fused to the site-directed modifying polypeptide, thus resulting in a chimeric polypeptide.

In some embodiments, a polynucleotide sequence encoding one or more components of a gene-regulating system described herein is operably linked to an inducible promoter. In some embodiments, a polynucleotide sequence encoding one or more components of a gene-regulating system described herein is operably linked to a constitutive promoter.

Methods of introducing polynucleotides and recombinant vectors into a host cell are known in the art, and any known method can be used to introduce components of a gene-regulating system into a cell. Suitable methods include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. pii: S0169-409X(12)00283-9), microfluidics delivery methods (See e.g., International PCT Publication No. WO 2013/059343), and the like. In some embodiments, delivery via electroporation comprises mixing the cells with the components of a gene-regulating system in a cartridge, chamber, or cuvette and applying one or more electrical impulses of defined duration and amplitude. In some embodiments, cells are mixed with components of a gene-regulating system in a vessel connected to a device (e.g., a pump) which feeds the mixture into a cartridge, chamber, or cuvette wherein one or more electrical impulses of defined duration and amplitude are applied, after which the cells are delivered to a second vessel.

In some embodiments, one or more components of a gene-regulating system, or polynucleotide sequence encoding one or more components of a gene-regulating system described herein are introduced to a cell in a non-viral delivery vehicle, such as a transposon, a nanoparticle (e.g., a lipid nanoparticle), a liposome, an exosome, an attenuated bacterium, or a virus-like particle. In some embodiments, the vehicle is an attenuated bacterium (e.g., naturally or artificially engineered to be invasive but attenuated to prevent pathogenesis including Listeria monocytogenes, certain Salmonella strains, Bifidobacterium longum, and modified Escherichia coli), bacteria having nutritional and tissue-specific tropism to target specific cells, and bacteria having modified surface proteins to alter target cell specificity. In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In some embodiments, the vehicle is a biological liposome. For example, the biological liposome is a phospholipid-based particle derived from human cells (e.g., erythrocyte ghosts, which are red blood cells broken down into spherical structures derived from the subject and wherein tissue targeting can be achieved by attachment of various tissue or cell-specific ligands), secretory exosomes, or subject-derived membrane-bound nanovescicles (30-100 nm) of endocytic origin (e.g., can be produced from various cell types and can therefore be taken up by cells without the need for targeting ligands).

In some embodiments, the methods of modified Tregs described herein comprise obtaining a population of Tregs from a sample. In some embodiments, a sample comprises a tissue sample, a fluid sample, a cell sample, a protein sample, or a DNA or RNA sample. In some embodiments, a tissue sample may be derived from any tissue type in the body including, but not limited to gut, skin, lung, liver, spleen, lymph nodes, and adipose tissue cell culture media comprising one or more populations of cells, buffered solutions comprising one or more populations of cells, and the like.

In some embodiments, the sample is processed to enrich or isolate a particular cell type, such as an Treg, from the remainder of the sample.

In some embodiments, the isolated Tregs are expanded in culture to produce an expanded population of Tregs. One or more activating or growth factors may be added to the culture system during the expansion process. For example, in some embodiments, one or more cytokines (such as TGF-β and/or IL-2) can be added to the culture system to enhance or promote cell proliferation and expansion. In some embodiments, one or more activating antibodies, such as an anti-CD3 antibody, may be added to the culture system to enhance or promote cell proliferation and expansion. In some embodiments, the Tregs may be co-cultured with feeder cells during the expansion process. In some embodiments, the methods provided herein comprise one or more expansion phases. Methods for ex vivo expansion of immune cells are known in the art, for example, as described in US Patent Application Publication Nos. 20180282694 and 20170152478 and U.S. Pat. Nos. 8,383,099 and 8,034,334.

At any point during the culture and expansion process, the gene-regulating systems described herein can be introduced to the Tregs to produce a population of modified Tregs. In some embodiments, the gene-regulating system is introduced to the population of Tregs immediately after enrichment from a sample. In some embodiments, the gene-regulating system is introduced to the population of Tregs before, during, or after the one or more expansion process. In some embodiments, the gene-regulating system is introduced to the population of Tregs immediately after enrichment from a sample or harvest from a subject, and prior to any expansion rounds. In some embodiments, the gene-regulating system is introduced to the population of Tregs after expansion.

In some embodiments, the modified Tregs produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

In some embodiments, the modified Tregs may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

### A. Producing Modified Regulatory T Cells Using CRISPR/Cas Systems

In some embodiments, a method of producing a modified Treg involves contacting a target DNA sequence with a complex comprising a gRNA and a Cas polypeptide. As discussed above, a gRNA and Cas polypeptide form a complex, wherein the DNA-binding domain of the gRNA targets the complex to a target DNA sequence and wherein the Cas protein (or heterologous protein fused to an enzymatically inactive Cas protein) modifies target DNA sequence. In some embodiments, this complex is formed intracellularly after introduction of the gRNA and Cas protein (or polynucleotides encoding the gRNA and Cas proteins) to a cell. In some embodiments, the nucleic acid encoding the Cas protein is a DNA nucleic acid and is introduced to the cell by transduction. In some embodiments, the Cas9 and gRNA components of a CRISPR/Cas gene editing system are encoded by a single polynucleotide molecule. In some embodiments, the polynucleotide encoding the Cas protein and gRNA component are comprised in a viral vector and introduced to the cell by viral transduction. In some embodiments, the Cas9 and gRNA components of a CRISPR/Cas gene editing system are encoded by different polynucleotide molecules. In some embodiments, the polynucleotide encoding the Cas protein is comprised in a first viral vector and the polynucleotide encoding the gRNA is comprised in a second viral vector. In some aspects of this embodiment, the first viral vector is introduced to a cell prior to the second viral vector. In some aspects of this embodiment, the second viral vector is introduced to a cell prior to the first viral vector. In such embodiments, integration of the vectors results in sustained expression of the Cas9 and gRNA components. However, sustained expression of Cas9 may lead to increased off-target mutations and cutting in some cell types. Therefore, in some embodiments, an mRNA nucleic acid sequence encoding the Cas protein may be introduced to the population of cells by transfection. In such embodiments, the expression of Cas9 will decrease over time, and may reduce the number of off target mutations or cutting sites.

In some embodiments, this complex is formed in a cell-free system by mixing the gRNA molecules and Cas proteins together and incubating for a period of time sufficient to allow complex formation. This pre-formed complex, comprising the gRNA and Cas protein and referred to herein as a CRISPR-ribonucleoprotein (CRISPR-RNP) can then be introduced to a cell in order to modify a target DNA sequence.

B. Producing Modified Regulatory T Cells Using shRNA Systems

In some embodiments, a method of producing a modified Treg introducing into the cell one or more DNA polynucleotides encoding one or more shRNA molecules with sequence complementary to the mRNA transcript of a target gene. The Treg can be modified to produce the shRNA by introducing specific DNA sequences into the cell nucleus via a small gene cassette. Both retroviruses and lentiviruses can be used to introduce shRNA-encoding DNAs into Tregs. The introduced DNA can either become part of the cell's own DNA or persist in the nucleus, and instructs the cell machinery to produce shRNAs. shRNAs may be processed by Dicer or AGO2-mediated slicer activity inside the cell to induce RNAi mediated gene knockdown.

### V. Compositions and Kits

The term “composition” as used herein refers to a formulation of a gene-regulating system or a modified Treg described herein that is capable of being administered or delivered to a subject or cell. Typically, formulations include all physiologically acceptable compositions including derivatives and/or prodrugs, solvates, stereoisomers, racemates, or tautomers thereof with any physiologically acceptable carriers, diluents, and/or excipients. A “therapeutic composition” or “pharmaceutical composition” (used interchangeably herein) is a composition of a gene-regulating system or a modified Treg capable of being administered to a subject for the treatment of a particular disease or disorder or contacted with a cell for modification of one or more endogenous target genes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In some embodiments, the present disclosure provides kits for carrying out a method described herein. In some embodiments, a kit can include:

(a) one or more nucleic acid molecules capable of reducing the expression or modifying the function of a gene product encoded by one or more endogenous target genes;

(b) one or more polynucleotides encoding a nucleic acid molecule that is capable of reducing the expression or modifying the function of a gene product encoded by one or more endogenous target genes;

(c) one or more proteins capable of reducing the expression or modifying the function of a gene product encoded by one or more endogenous target genes;

(d) one or more polynucleotides encoding a modifying protein that is capable of reducing the expression or modifying the function of a gene product encoded by one or more endogenous target genes;

(e) one or more gRNAs capable of binding to a target DNA sequence in an endogenous gene;

(f) one or more polynucleotides encoding one or more gRNAs capable of binding to a target DNA sequence in an endogenous gene;

(g) one or more site-directed modifying polypeptides capable of interacting with a gRNA and modifying a target DNA sequence in an endogenous gene;

(h) one or more polynucleotides encoding a site-directed modifying polypeptide capable of interacting with a gRNA and modifying a target DNA sequence in an endogenous gene;

(i) one or more guide DNAs (gDNAs) capable of binding to a target DNA sequence in an endogenous gene;

(j) one or more polynucleotides encoding one or more gDNAs capable of binding to a target DNA sequence in an endogenous gene;

(k) one or more site-directed modifying polypeptides capable of interacting with a gDNA and modifying a target DNA sequence in an endogenous gene;

(l) one or more polynucleotides encoding a site-directed modifying polypeptide capable of interacting with a gDNA and modifying a target DNA sequence in an endogenous gene;

(m) one or more gRNAs capable of binding to a target mRNA sequence encoded by an endogenous gene;

(n) one or more polynucleotides encoding one or more gRNAs capable of binding to a target mRNA sequence encoded by an endogenous gene;

(o) one or more site-directed modifying polypeptides capable of interacting with a gRNA and modifying a target mRNA sequence encoded by an endogenous gene;

(p) one or more polynucleotides encoding a site-directed modifying polypeptide capable of interacting with a gRNA and modifying a target mRNA sequence encoded by an endogenous gene;

(q) a modified Treg described herein; or

(r) any combination of the above.

In some embodiments, the kit comprises one or more components of a gene-regulating system (or one or more polynucleotides encoding the one or more components) and a reagent for reconstituting and/or diluting the components. In some embodiments, a kit comprising one or more components of a gene-regulating system (or one or more polynucleotides encoding the one or more components) and further comprises one or more additional reagents, where such additional reagents can be selected from: a buffer for introducing the gene-regulating system into a cell; a wash buffer; a control reagent; a control expression vector or RNA polynucleotide; a reagent for in vitro production of the gene-regulating system from DNA, and the like. Components of a kit can be in separate containers or can be combined in a single container.

In addition to above-mentioned components, in some embodiments a kit further comprises instructions for using the components of the kit to practice the methods of the present disclosure. The instructions for practicing the methods are generally recorded on a suitable recording medium. For example, the instructions may be printed on a substrate, such as paper or plastic, etc. As such, the instructions may be present in the kits as a package insert or in the labeling of the container of the kit or components thereof (i.e., associated with the packaging or sub-packaging). In other embodiments, the instructions are present as an electronic storage data file present on a suitable computer readable storage medium, e.g. CD-ROM, diskette, flash drive, etc. In other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### VI. Therapeutic Methods and Applications

In some embodiments, the modified Tregs and gene-regulating systems described herein may be used in a variety of therapeutic applications. For example, in some embodiments the modified Tregs and/or gene-regulating systems described herein may be administered to a subject for purposes such as gene therapy, e.g. to treat a disease, for use as an autoimmune disease therapeutic, or for biological research.

In some embodiments, the subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mice, rats, guinea pigs, hamsters, rabbits, etc.) may be used for experimental investigations.

Administration of the modified Tregs described herein, populations thereof, and compositions thereof can occur by injection, irrigation, inhalation, consumption, electro-osmosis, hemodialysis, iontophoresis, and other methods known in the art. In some embodiments, administration route is local or systemic. In some embodiments administration route is intraarterial, intracranial, intradermal, intraduodenal, intrammamary, intrameningeal, intraperitoneal, intrathecal, intratumoral, intravenous, intravitreal, ophthalmic, parenteral, spinal, subcutaneous, ureteral, urethral, vaginal, or intrauterine.

In some embodiments, the administration route is by infusion (e.g., continuous or bolus). Examples of methods for local administration, that is, delivery to the site of injury or disease, include through an Ommaya reservoir, e.g. for intrathecal delivery (See e.g., U.S. Pat. Nos. 5,222,982 and 5,385,582, incorporated herein by reference); by bolus injection, e.g. by a syringe, e.g. into a joint; by continuous infusion, e.g. by cannulation, such as with convection (See e.g., US Patent Application Publication No. 2007-0254842, incorporated herein by reference); or by implanting a device upon which the cells have been reversibly affixed (see e.g. US Patent Application Publication Nos. 2008-0081064 and 2009-0196903, incorporated herein by reference). In some embodiments, the administration route is by topical administration or direct injection. In some embodiments, the modified Tregs described herein may be provided to the subject alone or with a suitable substrate or matrix, e.g. to support their growth and/or organization in the tissue to which they are being transplanted.

In some embodiments, at least 1×10³ cells are administered to a subject. In some embodiments, at least 5×10³ cells, 1×10⁴ cells, 5×10⁴ cells, 1×10⁵ cells, 5×10⁵ cells, 1×10⁶, 2×10⁶, 3×10⁶, 4×10⁶, 5×10⁶, 1×10⁷, 1×10⁸, 5×10⁸, 1×10⁹, 5×10⁹, 1×10¹⁰, 5×10¹⁰, 1×10¹¹, 5×10¹¹, 1×10¹², 5×10¹², or more cells are administered to a subject. In some embodiments, between about 1×10⁷ and about 1×10¹² cells are administered to a subject. In some embodiments, between about 1×10⁸ and about 1×10¹² cells are administered to a subject. In some embodiments, between about 1×10⁹ and about 1×10¹² cells are administered to a subject. In some embodiments, between about 1×10¹⁰ and about 1×10¹² cells are administered to a subject. In some embodiments, between about 1×10¹¹ and about 1×10¹² cells are administered to a subject. In some embodiments, between about 1×10⁷ and about 1×10¹¹ cells are administered to a subject. In some embodiments, between about 1×10⁷ and about 1×10¹⁰ cells are administered to a subject. In some embodiments, between about 1×10⁷ and about 1×10⁹ cells are administered to a subject. In some embodiments, between about 1×10⁷ and about 1×10⁸ cells are administered to a subject. The number of administrations of treatment to a subject may vary. In some embodiments, introducing the modified Tregs into the subject may be a one-time event. In some embodiments, such treatment may require an on-going series of repeated treatments. In some embodiments, multiple administrations of the modified Tregs may be required before an effect is observed. The exact protocols depend upon the disease or condition, the stage of the disease and parameters of the individual subject being treated.

In some embodiments, the gene-regulating systems described herein are employed to modify cellular DNA or RNA in vivo, such as for gene therapy or for biological research. In such embodiments, a gene-regulating system may be administered directly to the subject, such as by the methods described supra. In some embodiments, the gene-regulating systems described herein are employed for the ex vivo or in vitro modification of a population of Tregs. In such embodiments, the gene-regulating systems described herein are administered to a sample comprising Tregs.

In some embodiments, the modified Tregs described herein are administered to a subject. In some embodiments, the modified Tregs described herein administered to a subject are autologous Tregs. The term “autologous” in this context refers to cells that have been derived from the same subject to which they are administered. For example, Tregs may be obtained from a subject, modified ex vivo according to the methods described herein, and then administered to the same subject in order to treat a disease. In such embodiments, the cells administered to the subject are autologous Tregs. In some embodiments, the modified Tregs, or compositions thereof, administered to a subject are allogenic Tregs. The term “allogeneic” in this context refers to cells that have been derived from one subject and are administered to another subject. For example, Tregs may be obtained from a first subject, modified ex vivo according to the methods described herein and then administered to a second subject in order to treat a disease. In such embodiments, the cells administered to the subject are allogenic Tregs.

In some embodiments, the modified Tregs described herein are administered to a subject in order to treat a disease. In some embodiments, treatment comprises delivering an effective amount of a population of cells (e.g., a population of modified Tregs) or composition thereof to a subject in need thereof. In some embodiments, treating refers to the treatment of a disease in a mammal, e.g., in a human, including (a) inhibiting the disease, i.e., arresting disease development or preventing disease progression; (b) relieving the disease, i.e., causing regression of the disease state or relieving one or more symptoms of the disease; and (c) curing the disease, i.e., remission of one or more disease symptoms. In some embodiments, treatment may refer to a short-term (e.g., temporary and/or acute) and/or a long-term (e.g., sustained) reduction in one or more disease symptoms. In some embodiments, treatment results in an improvement or remediation of the symptoms of the disease. The improvement is an observable or measurable improvement, or may be an improvement in the general feeling of well-being of the subject.

The effective amount of a modified Treg administered to a particular subject will depend on a variety of factors, several of which will differ from patient to patient including the disorder being treated and the severity of the disorder; activity of the specific agent(s) employed; the age, body weight, general health, sex and diet of the patient; the timing of administration, route of administration; the duration of the treatment; drugs used in combination; the judgment of the prescribing physician; and like factors known in the medical arts.

In some embodiments, an effective amount of modified Tregs will be at least 1×10³ cells, for example 5×10³ cells, 1×10⁴ cells, 5×10⁴ cells, 1×10⁵ cells, 5×10⁵ cells, 1×10⁶, 2×10⁶, 3×10⁶, 4×10⁶, 5×10⁶, 1×10⁷, 1×10⁸, 5×10⁸, 1×10⁹, 5×10⁹, 1×10¹⁰, 5×10¹⁰, 1×10¹¹, 5×10¹¹, 1×10¹², 5×10¹², or more cells.

In some embodiments, the modified Tregs and gene-regulating systems described herein may be used in the treatment an autoimmune disorder. Unless stated otherwise, the terms “disorder” and “disease” are used interchangeably herein. The term “autoimmune disorder” as used herein is a disease or disorder arising from and directed against an individual's own tissues or organs or a co-segregate or manifestation thereof or resulting condition therefrom. Autoimmune diseases are primarily caused by dysregulation of adaptive immune responses and autoantibodies or autoreactive T cells against self structures are formed.

Exemplary autoimmune disorders include autoimmune hepatitis, inflammatory bowel disease (IBD), Crohn's disease, colitis, ulcerative colitis, type 1 diabetes, alopecia areata, vasculitis, temporal arthritis, lupus, celiac disease, Sjogrens syndrome, polymyalgia rheumatica, multiple sclerosis, arthritis, rheumatoid arthritis, graft versus host disease (GVHD) and psoriasis.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### EXAMPLES

### Example 1: Materials and Methods

The experiments described herein utilize the CRISPR/Cas9 system to modulate expression of endogenous target genes in regulatory T cells (Treg) for their clinical use as an immunotherapy for the treatment of autoimmune disease.

### I. Materials

gRNAs: Unless otherwise indicated, all experiments use single-molecule gRNAs (sgRNAs). Dual gRNA molecules were used as indicated and were formed by duplexing 200 μM tracrRNA (IDT Cat #1072534) with 200 μM of target-specific crRNA (IDT) in nuclease free duplex buffer (IDT Cat #11-01-03-01) for 5 min at 95° C., to form 100 μM of tracrRNA:crRNA duplex, where the tracrRNA and crRNA are present at a 1:1 ratio.

Cas9: Cas9 was expressed in target cells by introduction of either Cas9 mRNA or a Cas9 protein. Unless otherwise indicated, Cas9-encoding mRNA comprising a nuclear localization sequence (Cas9-NLS mRNA) derived from S. pyogenes (Trilink L-7206) or Cas9 protein derived from S. pyogenes (IDT Cat #1074182) was used in the following experiments.

RNPs: gRNA-Cas9 ribonucleoproteins (RNPs) were formed by combining 1.2 μL of 100 M tracrRNA:crRNA duplex with 1 μL of 20 μM Cas9 protein and 0.8 μL of PBS. Mixtures were incubated at RT for 20 minutes to form the RNP complexes.

Lentiviral Expression Constructs: A library of 56,408 sgRNAs each targeting a single gene in the human genome was cloned into an expression vector containing the human U6 promotor. In total, 5,137 genes were targeted by this library of gRNAs. The plasmids further comprised an EF1L promotor driving expression of RFP, a T2A sequence, and puromycin resistance cassette.

Lentiviruses encoding the sgRNA library described above were generated as follows. Briefly, 578×10⁶ of LentiX-293T cells were plated in a 10-layer CellSTACK 24 hours prior to transfection. Serum-free OptiMEM, TransIT-293, and helper plasmids (116 μg VSVG and 231 μg PAX2-Gag-Pol) were combined with 462 μg of sgRNA-expressing plasmids described above and incubated for 5 minutes. This mixture was added to the LentiX-293T cells with fresh media. Media was replaced 18 hours after transfection and viral supernatants were collected 48 hours post-transfection. Supernatants were treated with Benzonase® nuclease and passed through a 0.45 μm filter to isolate the viral particles. Virus particles were then concentrated by Tangential Flow Filtration (TFF), aliquoted, tittered, and stored at −80° C.

### II. Methods

Human Treg cell Isolation: Peripheral blood Treg and CD4+T effector (Teff) cells were isolated from fresh leukopacks or whole blood from healthy volunteer blood donors in a step-wise fashion. First, peripheral blood mononuclear cells (PBMCs) were obtained by Ficoll gradient centrifugation. Next, CD4+ T cells were isolated via negative immunomagnetic selection using EasySep Human CD4+ T Cell Isolation Kit (StemCell Technologies, Cat #17952). For enrichment of Tregs, isolated CD4+ T cells were further labeled with a monoclonal antibody against CD25-PE followed by positive selection using EasySep Human PE Positive Selection Kit (StemCell Technologies, Cat #18561). Enriched CD4+CD25+ cells were subsequently labeled with monoclonal antibodies specific for CD4 and CD127 prior to fluorescence activated cell sorting (FACS) to obtain a pure population of Tregs. Tregs were sorted based on the following parameters: CD4+CD25^{high}CD127^{dim}.

Human Treg cell expansion ex vivo: Isolated Tregs were plated at 2×10⁶ cells/mL in X-VIVO 15 T Cell Expansion Medium (Lonza, Cat #04-418Q) supplemented with human inactivated serum AB (10%) and human IL-2 (60 ng/ml or 300 units/ml). On day 0 and day 10 of culture, anti-CD3/CD28 Treg expander beads or Immunocult Human CD3/CD28/CD2 T-cell Activators (tetrameric) were added to the culture at a 1:4 or 1:1 cell:bead ratio, respectively, or in a 1× fashion in the case of the activators. Additional human IL-2 was supplemented to the culture every 2-3 days.

Lentiviral transduction of Treg cells: Following 10 days of expansion, Treg were re-activated using anti-CD3/CD28 Treg expander beads for 18 hours prior to being seeded at 5×10⁶ cells per well in a 6 well plate, in 1.5 mL volume of X-VIVO 15 media, 6 ng/mL human IL-2. After the same expansion, Teff were re-activated using Immunocult Human CD3/CD28/CD2 T-cell Activators for 18 hours prior to being seeded at 5×10⁶ cells per well in a 6 well plate in 1.5 mL volume of X-VIVO 15 media, 10 ng/mL human IL-2. Lentivirus expressing sgRNA library was added separately to both cell types at an MOI capable of infecting 80% of all cells. 20 μL of Retronectin (1 mg/mL) was added to each well. X-VIVO 15 media was added to a final volume of 2.0 mL per well. Plates were spun at 600×g for 1.5 hours at room temperature. After 18 hours (day 2), cells were washed and seeded at 1×10⁶ cells/mL in X-VIVO 15. To Treg cultures, 60 ng/mL IL2 was added and to Teff cultures, 10 ng/mL IL2 and T-cell activators were added.

Electroporation of T cells: Where indicated, gRNAs and/or Cas9 were introduced to Treg cells by electroporation. For example, where Treg cells were transduced with a lentivirus expressing specific sgRNAs, Cas9 mRNA can be electroporated into the cells after transduction. Alternatively, dual gRNA duplexes can be complexed with a Cas9 protein to form an RNP, which can then be electroporated into Treg cells. The electroporation protocol for either Cas9 mRNA or RNPs is as follows.

Three days after Treg and Teff cell re-activation (day 13 of expansion), Treg and Teff cells transduced with lentivirus expressing specific sgRNAs were harvested and resuspended in nucleofection buffer (18% supplement 1, 82% P3 buffer from the Amaxa P3 primary cell 4D-Nuclefector X kit S (Cat #V4XP-3032)) at a concentration of 100×10⁶ cells/mL. 4 μg (4 μL of 1 mg/mL) of S. pyogenes Cas9-NLS mRNA was added to the cell mixture per 20 μL of cell solution and 24 μL of the cell/mRNA mixture was then added to each reaction well. Cells were electroporated following the “T cell, Human, Stim” program (EO-115). After electroporation, 80 L of warm X-VIVO 15 media was added to each well, and cells were pooled into a culture flask at a density of 2×10⁶ cells/mL in X-VIVO 15 media containing IL-2 (Treg: 60 ng/mL; Teff 10 ng/mL). On day 4 after reactivation, cells were washed, counted, and utilized for functional assays, as described below. Editing efficiency of target genes were determined by FACS analysis of surface or intracellular proteins (e.g., CD45, Foxp3) and/or TIDE/NGS analysis of the genomic cut-site.

Editing of a gene is assessed by next generation sequencing. For this method, genomic DNA (gDNA) was isolated from edited T cells using the Qiagen Blood and Cell Culture DNA Mini Kit (Cat #: 13323) following the vendor recommended protocol and quantified. Following gDNA isolation, PCR was performed to amplify the region of edited genomic DNA using locus-specific PCR primers containing overhangs required for the addition of Illumina Next Generation sequencing adapters. The resulting PCR product was run on a 1% agarose gel to ensure specific and adequate amplification of the genomic locus occurred before PCR cleanup was conducted according to the vendor recommended protocol using the Monarch PCR & DNA Cleanup Kit (Cat #: T1030S). Purified PCR product was then quantified, and a second PCR was performed to anneal the Illumina sequencing adapters and sample specific indexing sequences required for multiplexing. Following this, the PCR product was run on a 1% agarose gel to assess size before being purified using AMPure XP beads (produced internally). Purified PCR product was then quantified via qPCR using the Kapa Illumina Library Quantification Kit (Cat #: KK4923) and Kapa Illumina Library Quantification DNA Standards (Cat #: KK4903). Quantified product was then loaded on the Illumina NextSeq 500 system using the Illumina NextSeq 500/550 Mid Output Reagent Cartridge v2 (Cat #: FC-404-2003). Analysis of produced sequencing data was performed to assess insertions and deletions (indels) at the anticipated cut site in the DNA of the edited T cell pool.

Immunophenotyping and TSDR analysis of edited Treg cells: Four days after editing of Treg cells, cells were labeled with CellTrace Violet reagent to track cell division and stimulated with anti-CD3/CD28 Treg expander beads in the presence of human IL-2 (500 units/ml). After four days of stimulation, cells were restimulated with eBioscience Cell Stimulation Cocktail (plus protein transport inhibitors) (eBioscience, Cat #: 00-4975-03) for 5 hours. Cell surface staining was performed with the following antibodies: anti-CD4 (SK3), -CD25 (MA-251), -TNFRSF4 (Ber-ACT35), and CD45 (HI30). Staining was performed for 20 minutes at 4° c. in the presence of human FcBlock reagent (BD Biosciences, Cat #564219). To detect intracellular proteins, after surface staining, cells were fixed and permeabilized using eBioscience Foxp3/Transcription Factor Staining Buffer Set (eBioscience, Cat #: 00-5523-00) and stained with the following antibodies: anti-Foxp3 (259D/C7), Helios (22F6), and IL-10 (JES3-9D7). The LSRFortessa (BD Biosciences) was used for data collection and analysis was performed using FlowJo software (TreeStar). For TSDR analysis, genomic DNA was isolated from edited Tregs as described previously using the Qiagen Blood and Cell Culture DNA Mini Kit (Cat #: 13323) following the vendor recommended protocol. Bisulfite conversion and pyrosequencing of genomic DNA was performed by EpigenDx (assay ID ADS783-FS2) to quantify the methylation status of the FOXP3 gene region.

In vitro suppression of allogeneic T effector cells by edited Tregs: The suppressive function of Tregs was determined using a modified version of a method developed by Collison et al. (“In vitro Treg suppression assays,” Methods Mol Biol. 707:21-37 (2011)). Frozen sgRNA-edited Tregs and unedited allogenic effector T cells (hereafter referred to as T responder cells) were thawed and rested overnight in X-VIVO 15 T Cell Expansion media (Lonza, Cat #04-418Q) supplemented with 10% inactivated male human sera and 600 units/ml IL-2. T responder cells and Tregs were washed in PBS containing 0.1% BSA and then incubated in the same buffer containing 10 μM CellTrace Violet or 4 μM CFSE, respectively, for 10 minutes at room temperature. Labeled T responder cells were resuspended in T cell expansion media and seeded at 50,000 cells (50 μl) per well in a 96 well U-bottom plate. On a separate plate, labelled Tregs, resuspended in T cell expansion media, were seeded at 50,000 cells (50 μl) per well, serially diluted, and then mixed with T responder cells at ratios between 1:2 to 1:32. Finally, 100 μl of 0.5 μl/ml ImmunoCult™ Human CD3/CD28 T Cell Activators were added to each well. Wells without Tregs or CD3/28 tetramers served as positive and negative controls, respectively. After 4 days of incubation at 37° C., cells were stained with antibodies to CD4, CD3, Foxp3, and Helios (described above). Data was acquired on a BD LSRFortessa X-20 cell analyzer (BD Biosciences), and the proliferation of T responder cells was analyzed using FlowJo (TreeStar Inc.). Treg suppression was determined as: suppression (%):100−[100×(% proliferating cells with Tregs)/(% proliferating cells without Tregs).

Assessment of edited Tregs function in vivo: The ability of CRISPR edited human Tregs to reduce autoimmune responses was evaluated in the NSG-human PBMC xenogeneic mouse model of Graft versus Host Disease (GvHD). A model previously described by Cuende et al. was adapted (“Monoclonal antibodies against GARP/TGF-β1 complexes inhibit the immunosuppressive activity of human regulatory T cells in vivo,” Sci Transl Med. 7(284):284ra56 (2015)) to be modulated by the transfer of human Tregs. Female NCG mice (8 to 12 weeks old) were injected intravenously with 20×10⁶ human peripheral blood mononuclear cells (PBMCs). Fourteen days later, mice were randomized by bodyweight into four groups of five animals per group, and three groups were intravenously dosed with 2×10⁶ edited human Tregs. One group served as an untreated control and did not receive Treg treatment. Prior to treatment, the human Tregs were edited by electroporation with gRNA/Cas9 RNP complexes comprising (1) a control gRNA targeting the OR1A1 gene (SEQ ID NO: 1 GCTGACCAGTAACTCCCAGG); (2) a single gRNA targeting the PRDM1 gene (SEQ ID NO: 2 TTGGACAGATCTATTCCAGA); and (3) a single gRNA targeting the TNFRSF4 gene (SEQ ID NO: 3 GGATGTGCGTGGGGGCTCGG). Editing efficiency of the gRNA/Cas9 complex targeting the PRDM1 and TNFRSF4 genes was assessed by next-generation sequencing and determined to be 99% and 83%, respectively. Body weight and GvHD score (the sum of the scores given for weight loss, activity, posture, fur texture, and skin integrity) was measured three times per week after Treg transfer. Flow cytometry was also performed on peripheral blood samples obtained fifteen days post-Treg transfer to track CD8+T effector cell proliferation and activation. The results are discussed in Example 4.

### Example 2: Identification of Targets for Immunomodulation of Treg Cells Through In Vitro Crispr/Cas9 Functional Genomic Screens

Experiments were performed to identify targets that modulate the fitness of Tregs during in vitro expansion. A pooled, genome-wide CRISPR screen was performed in which a pool of sgRNAs, each of which target a single gene, was introduced into a population human Treg cells or donor-matched Teff cells, such that each cell in the population comprised a single sgRNA targeting a single gene. To determine the effect of a particular gene on Tregs (or Teff cells) during ex vivo expansion, the frequency of each sgRNA in the population of Treg (or Teff cells) was determined at the beginning of the experiment and compared to the frequency of the same sgRNA at a later time-point in the experiment. The frequency of sgRNAs targeting genes that positively regulate Treg (or Teff cells) expansion in vitro (e.g., genes that positively-regulate Treg (or Teff cells) proliferation or viability) is expected to increase over time, while the frequency of sgRNAs targeting genes that negatively regulate Treg (or Teff cells) expansion in vitro (e.g., genes that negatively-regulate Treg (or Teff cells) proliferation or viability) is expected to decrease over time.

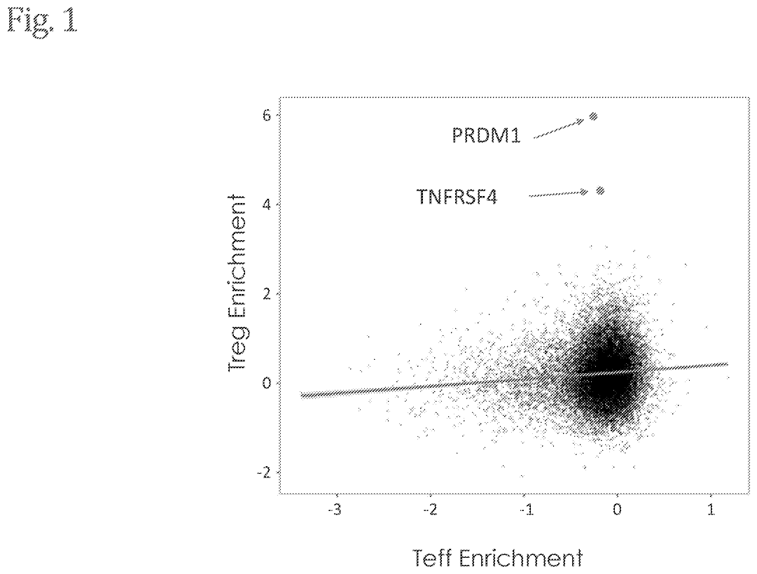

The distribution and/or frequency of each sgRNA in the aliquots taken at various time points during in vitro expansion was analyzed and compared to the distribution and/or frequency of each sgRNA in the initial edited Treg (or Teff cells) population. Statistical analyses were performed for each individual sgRNA to identify sgRNAs that were significantly enriched in Treg (or Teff cells) populations after in vitro expansion and to assign an enrichment score to each of the guides. For each individual sgRNA in our screening library, an enrichment score was calculated by taking the ratio of guide counts observed at the screen endpoint and dividing by the number of reads observed for that guide at the beginning of the screen. To calculate a gene-level enrichment score, an aggregate enrichment score was calculated as the median sgRNA enrichment score. To calculate the statistical significance of the gene-level enrichment a nominal p-value was calculated for each guide as the percentile for enrichment of that guide relative to all other guides in the library. These p-values were combined using the logit p-value combination method (Mudholkar 1977), generating an aggregate gene-level p-value for target enrichment. Gene-level p-values were corrected for multiple-testing using the Benjamini-Hochberg procedure. To identify target genes that have a consistent and reproducible effect on Treg (or Teff cells) accumulation in vitro across multiple sgRNAs, a false-discovery-rate (FDR) cutoff of equal to or less than 0.2 was set. The results of these experiments are shown below in Table 2 and FIG. 1. Genes in Table 2 are the genes with the top-10 gene-level enrichment scores; PRDM1 and TNFRSF4 were the two genes that passed the FDR criteria.

TABLE 2
Target Genes Identified by Percentile Scores

| Target Name | Enrichment | FDR |
|---|---|---|
| PRDM1 | 6.84 | 4.5E−7 |
| TNFRSF4 | 5.34 | 1.3E−3 |
| REEP3 | 3.53 | 0.43 |
| MRPL32 | 3.27 | 0.47 |
| FSCN3 | 3.26 | 0.46 |
| KLC3 | 2.75 | 0.48 |
| C4BPA | 2.67 | 0.48 |
| LZTS1 | 2.64 | 0.43 |
| CDK16 | 2.60 | 0.43 |
| ADNP | 2.56 | 0.43 |

### Example 3: Validation of Targets for Immunomodulation of Treg Cells

Targets with an FDR cutoff equal to less than 0.2 were selected for further evaluation in a single-guide format to determine whether editing a target gene in Treg cells altered the stability and/or function of these cells. Evaluation of exemplary targets is described herein, however these methods can be used to evaluate any of the potential targets described above.

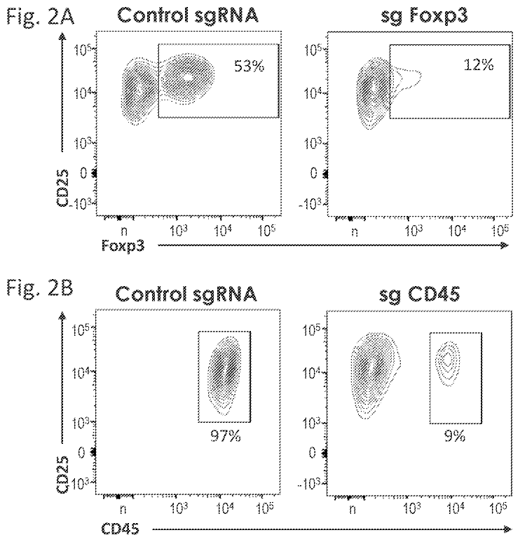

Immunophenotyping of edited human Treg cells: Human Treg cells were isolated and expanded ex vivo as described above and edited by electroporation using guide RNAs complexed to Cas9 in an RNP format for individual target genes. As shown in FIG. 2, high efficiency of editing of target genes could be achieved using the methods described. The consequence of editing of individual target genes identified through our in vitro CRISPR/Cas9 functional genomics screen in human Treg cells were determined by flow cytometry to quantify on a per-cell basis, any alterations in the proliferative capacity and in the frequency and magnitude of specific transcription factors and cytokines known to be important for Treg stability and function.

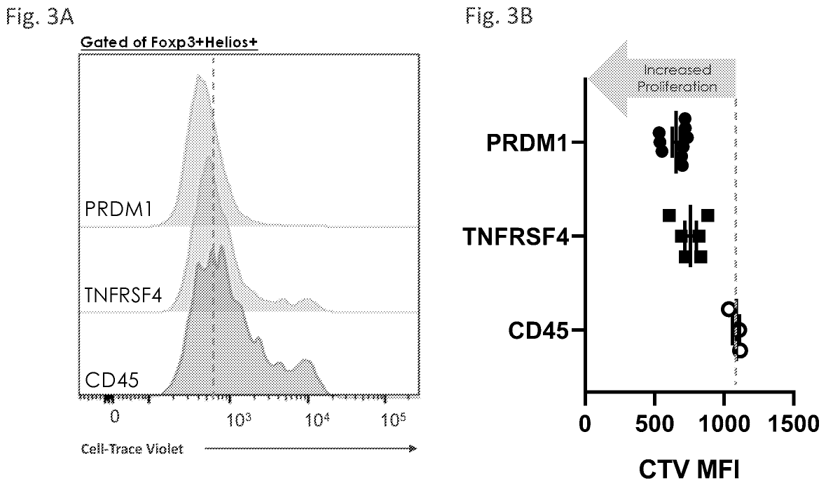

Edited Treg cells were restimulated with anti-CD3/CD28 Treg expander beads and proliferative capacity, transcription factor expression, and cytokine production were assessed at day 4. As shown in FIG. 3, PRDM1- and TNFRSF4-edited Tregs demonstrated a 40% and 30% decrease, respectively, in the mean fluorescence intensity (MFI) of CTV staining compared to control, CD45-edited Treg cells. A reduction in CTV staining occurs during each round of cell division, thus the reduction in CTV staining observed in edited Treg cells is indicative of increased proliferation of PRDM1- and TNFRSF4-edited Treg cells.

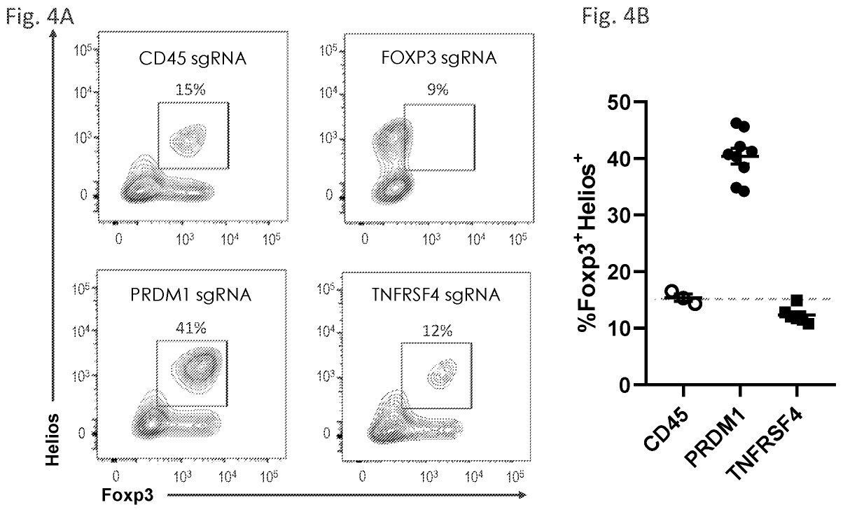

The transcription factor Helios in Treg cells is known to be essential for the stability of Treg cells (Kim H J, Barnitz R A, Kreslavsky T, et al. Stable inhibitory activity of regulatory T cells requires the transcription factor Helios. Science. 2015; 350(6258):334-9.). Further, binding of Helios with the Treg lineage-determining transcription factor, Foxp3, is strongly associated with the expression of core Treg signature genes (Kwon H K, Chen H M, Mathis D, Benoist C. Different molecular complexes that mediate transcriptional induction and repression by FoxP3. Nat Immunol. 2017; 18(11):1238-1248). Thus, the co-expression of Helios and Foxp3 in Treg cells has been associated with improved stability and increased immunosuppressive function. As shown in FIG. 4, editing of PRDM1 in Treg cells led to a 2.6-fold increase in the proportion Treg cells co-expressing both Foxp3 and Helios demonstrating that editing of PRDM1 leads to phenotypic alterations in Treg cells that associated with improved stability and immunosuppressive function of Treg cells.

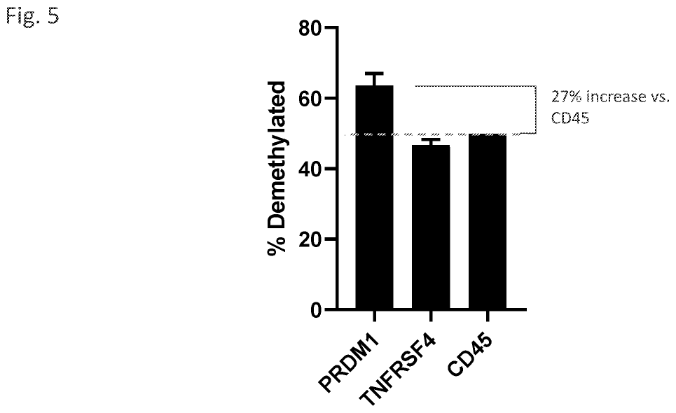

In addition, several studies have shown that demethylation of a conserved region within the Foxp3 locus named Treg-specific demethylated region (TSDR) is required to maintain expression of Foxp3 in the progeny of dividing Treg cells (Zheng et al. “Role of conserved non-coding DNA elements in the Foxp3 gene in regulatory T-cell fate,” Nature 463:808-12 (2010); Polansky J K, et al., “DNA methylation controls Foxp3 gene expression,” Eur. J. Immunol. 38: 1654-1663 (2008)). As shown in FIG. 5, editing of TNFRSF4 did not affect the TSDR methylation status in Treg cells, and editing of PRDM1 increased the demethylation of TSDR (by 27% as compared to CD45-edited control T reg cells). These data indicate that editing of PRDM1 drives accumulation of stable Treg cells.

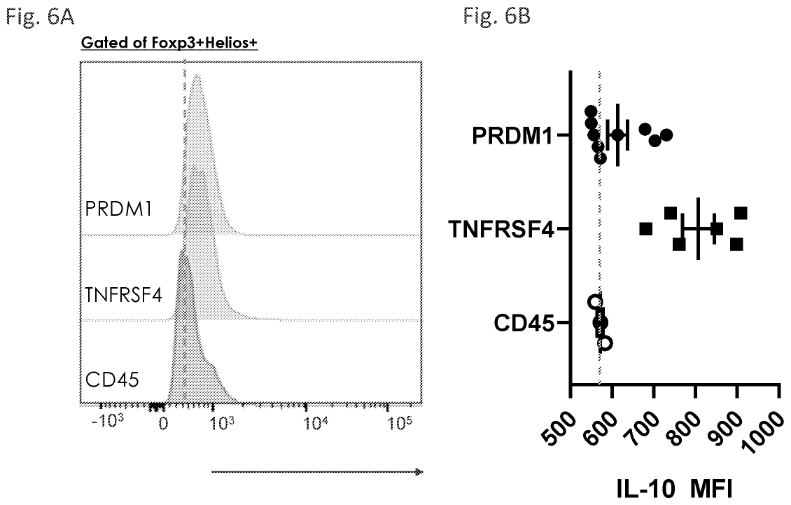

The production of immunosuppressive cytokines, such as IL-10, by Treg cells is a major mechanism whereby Treg cells are able to mediate their suppressive function. Indeed, Treg cells that are unable to produce IL-10 are unable to prevent effector T cell-mediated inflammation (Asseman C, Mauze S, Leach M W, Coffmnan R L, Powrie F. An essential role for interleukin 10 in the function of regulatory T cells that inhibit intestinal inflammation. J Exp Med. 1999; 190(7):995-1004). As shown in FIG. 6, editing of TNFRSF4 in Treg cells led to a 40% increase in the capacity of Treg cells to produce IL-10 compared to CD45-edited control Treg cells. Editing of PRDM1 led to a 10% increase in IL-10 production in Treg cells. Similar experiments demonstrated that editing of TNFRSF4 did not impact pro-inflammatory cytokines, including IL-17A and IFNγ.

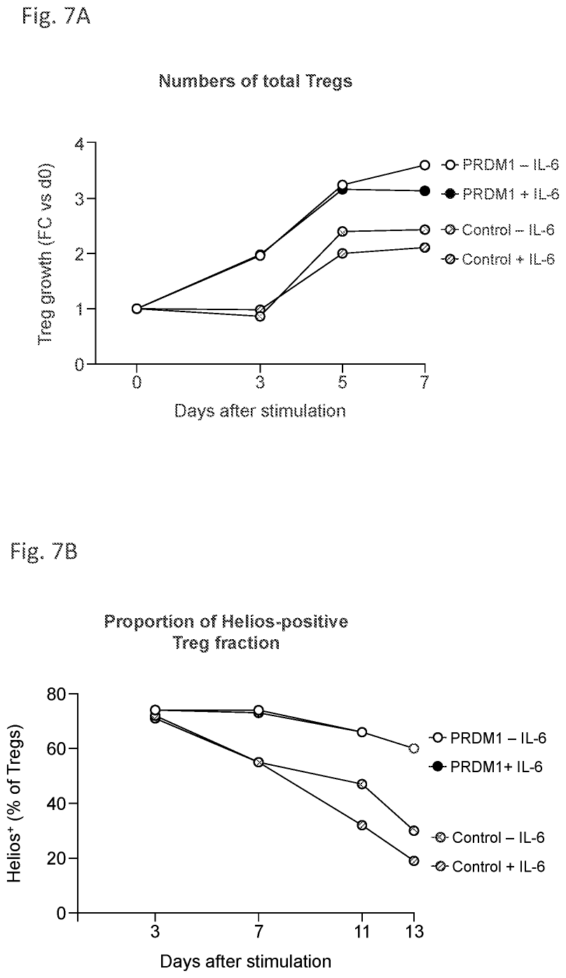

Inflammatory cytokines, such as IL-6, can destabilize Tregs and weaken their suppressive function (Yang et al., “Molecular antagonism and plasticity of regulatory and inflammatory T cell programs,” Immunity 29:44-56 (2008)). In mice, the destabilization of Tregs by IL-6 is accelerated in the absence of PRDM1 (Garg et al., “Blimp1 Prevents Methylation of Foxp3 and Loss of Regulatory T Cell Identity at Sites of Inflammation,” Cell Reports 26: 1854-1868 (2019)). To determine whether IL-6 drives destabilization of PRDM1-deficient human Tregs, PRDM1- and control-edited Tregs were cultured in the presence or absence of 50 ng/ml IL-6. The results, shown in FIGS. 7A and B, demonstrate that, in contrast to mouse Tregs, PRDM1-edited human Tregs maintain stability, as indicated by their high expression of Helios, even in the presence of high amounts of IL-6.

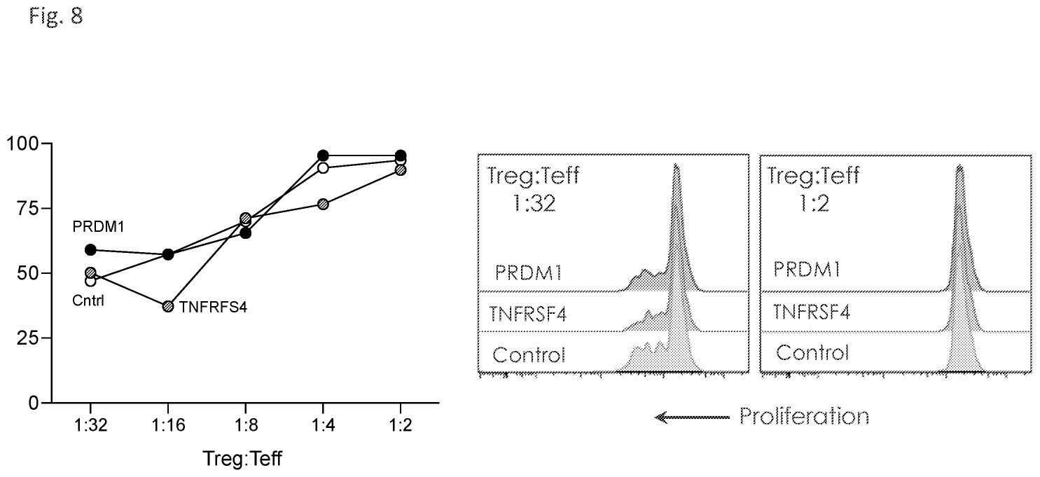

The suppressive function of Tregs is dependent on various metabolic processes, some of which are down-regulated as the Tregs undergo proliferation in vitro (Thornton A M, et al., “CD4+CD25+ immunoregulatory T cells suppress polyclonal T cell activation in vitro by inhibiting interleukin 2 production,” J Exp Med. 188:287-96 (1998); Kuniyasu Y, et al., “Naturally anergic and suppressive CD25(+) CD4(+) T cells as a functionally and phenotypically distinct immunoregulatory T cell subpopulation,” Int Immunol. 12:1145-55 (2000)). Given that editing of PRDM1 and TNFRSF4 led to increased proliferation of Tregs, the ability of such Tregs to suppress effector T cell proliferation was assessed. In an in-vitro-co-culture system (see Methods) in which Tregs are cultured for 4 days together with labeled effector T cells at ratios between 1:2 to 1:32, both PRDM1- and TNFRSF4-edited Tregs were able to suppress the proliferation of effector T cells, and their suppressive function was comparable to that of control-edited Tregs (FIG. 8).

Taken together, these data demonstrate that, editing of Treg cells with individual guide sequences to genes identified via our CRISPR/Cas9 functional genomic screen leads to distinct alterations in the proliferative capacity and in the frequency and magnitude of specific transcription factors and cytokines known to be important for Treg stability and function.

### Example 4: Validation of Targets for Immunomodulation of Treg Cells

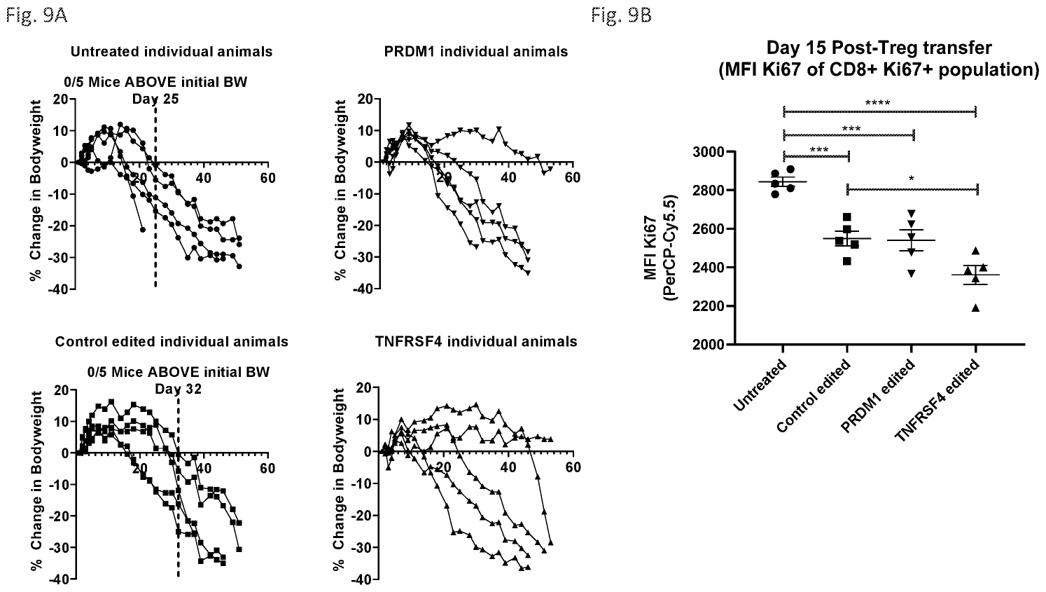

The data in FIG. 9A shows that human Treg-treated mice undergoing GvHD have enhanced survival versus untreated mice. The time for all five untreated mice to drop below their initial bodyweight was 25 days, versus 32 days for control edited Treg treated mice. The TNFRSF4−/− Treg treated group had a mouse maintain weight above the initial measurement to day 58 post-Treg transfer (72 days post PBMC transfer). FIG. 9B shows flow cytometry data on peripheral blood from mice on day fifteen post-Treg transfer. Ki67 staining intensity has been demonstrated to be a surrogate marker to quantify the proliferative capacity of cells (Miller et al. (“Ki67 is a Graded Rather than a Binary Marker of Proliferation versus Quiescence,” Cell Rep. 24(5):1105-1112.e5 (2018)). Ki67 staining intensity was reduced on human CD8 cells in all groups where Tregs were transferred, demonstrating that Tregs were capable of suppressing inflammation. Further, mice treated with TNFRSF4-edited Tregs were found to be further reduced in Ki67 staining intensity within their CD8+ T cell population, demonstrating that loss of TNFRSF4 leads to more potent Tregs in vivo (FIG. 9B).

1. A modified regulatory T cell (Treg) comprising a gene-regulating system capable of reducing expression and/or function of one or more endogenous target genes comprising TNFRSF4,
wherein the reduced expression and/or function of the one or more endogenous genes enhances an immunosuppressive function of the Treg.

2. The modified Treg of claim 1, wherein the gene-regulating system comprises (i) a nucleic acid molecule; (ii) an enzymatic protein; or (iii) a nucleic acid molecule and an enzymatic protein.

3. The modified Treg of claim 2, wherein the gene-regulating system comprises a nucleic acid molecule selected from an siRNA, an shRNA, a microRNA (miR), an antagomiR, or an antisense RNA.

4. The modified Treg of claim 2, wherein the gene-regulating system comprises an enzymatic protein, and wherein the enzymatic protein has been engineered to specifically bind to a target sequence in one or more of the endogenous genes.

5. The modified Treg of claim 4, wherein the protein is a Transcription activator-like effector nuclease (TALEN), a zinc-finger nuclease, or a meganuclease.

6. The modified Treg of claim 2, wherein the gene-regulating system comprises a nucleic acid molecule and an enzymatic protein, wherein the nucleic acid molecule is a guide RNA (gRNA) molecule and the enzymatic protein is a Cas protein or Cas ortholog.

7. The modified Treg of claim 6, wherein the Cas protein is a Cas9 protein.

8. The modified Treg of claim 6, wherein the Cas protein is a wild-type Cas protein comprising two enzymatically active domains, and capable of inducing double stranded DNA breaks.

9. The modified Treg of claim 6, wherein the Cas protein is a Cas nickase mutant comprising one enzymatically active domain and capable of inducing single stranded DNA breaks.

10. The modified Treg of claim 6, wherein the Cas protein is a deactivated Cas protein (dCas) and is associated with a heterologous protein capable of modulating the expression of the one or more endogenous target genes.

11. The modified Treg of claim 10, wherein the heterologous protein is selected from the group consisting of MAX-interacting protein 1 (MXI1), Krüppel-associated box (KRAB) domain, methyl-CpG binding protein 2 (MECP2), and four concatenated mSin3 domains (SID4X).

12. A modified Treg comprising a gene-regulating system capable of reducing expression and/or function of one or more endogenous target genes comprising PRDM1,
wherein the reduced expression and/or function of the one or more endogenous genes enhances an immunosuppressive function of the Treg.

13. The modified Treg of claim 12, wherein the gene-regulating system comprises (i) a nucleic acid molecule; (ii) an enzymatic protein; or (iii) a nucleic acid molecule and an enzymatic protein.

14. The modified Treg of claim 13, wherein the gene-regulating system comprises a nucleic acid molecule selected from an siRNA, an shRNA, a microRNA (miR), an antagomiR, or an antisense RNA.

15. The modified Treg of claim 13, wherein the gene-regulating system comprises an enzymatic protein, and wherein the enzymatic protein has been engineered to specifically bind to a target sequence in one or more of the endogenous genes.

16. The modified Treg of claim 15, wherein the protein is a Transcription activator-like effector nuclease (TALEN), a zinc-finger nuclease, or a meganuclease.

17. The modified Treg of claim 13, wherein the gene-regulating system comprises a nucleic acid molecule and an enzymatic protein, wherein the nucleic acid molecule is a guide RNA (gRNA) molecule and the enzymatic protein is a Cas protein or Cas ortholog.

18. The modified Treg of claim 17, wherein the Cas protein is a Cas9 protein.

19. The modified Treg of claim 17, wherein the Cas protein is a wild-type Cas protein comprising two enzymatically active domains, and capable of inducing double stranded DNA breaks.

20. The modified Treg of claim 17, wherein the Cas protein is a Cas nickase mutant comprising one enzymatically active domain and capable of inducing single stranded DNA breaks.

21. The modified Treg of claim 17, wherein the Cas protein is a deactivated Cas protein (dCas) and is associated with a heterologous protein capable of modulating the expression of the one or more endogenous target genes.

22. The modified Treg of claim 21, wherein the heterologous protein is selected from the group consisting of MAX-interacting protein 1 (MXI1), Krüppel-associated box (KRAB) domain, methyl-CpG binding protein 2 (MECP2), and four concatenated mSin3 domains (SID4X).

23. The modified Treg of any one of claims 1-22, wherein the gene-regulating system is capable of reducing the expression and/or function of at least 2, 3, 4, 5, 6 or more of endogenous target genes.

24. A modified Treg comprising a gene-regulating system capable of reducing the expression and/or function of one or more endogenous target genes selected from the group consisting of TNFRSF4, PRDM1, REEP3, MRPL32, FSCN3, KLC3, C4BPA, LZTS1, CDK16, and ADNP,
wherein the reduced expression and/or function of the one or more endogenous genes enhances an immunosuppressive function of the Treg.

25. The modified Treg of claim 24, wherein the gene-regulating system comprises (i) a nucleic acid molecule; (ii) an enzymatic protein; or (iii) a nucleic acid molecule and an enzymatic protein.

26. The modified Treg of claim 24, wherein the gene-regulating system comprises a nucleic acid molecule selected from an siRNA, an shRNA, a microRNA (miR), an antagomiR, or an antisense RNA.

27. The modified Treg of claim 24, wherein the gene-regulating system comprises an enzymatic protein, and wherein the enzymatic protein has been engineered to specifically bind to a target sequence in one or more of the endogenous genes.

28. The modified Treg of claim 27, wherein the protein is a Transcription activator-like effector nuclease (TALEN), a zinc-finger nuclease, or a meganuclease.

29. The modified Treg of claim 24, wherein the gene-regulating system comprises a nucleic acid molecule and an enzymatic protein, wherein the nucleic acid molecule is a guide RNA (gRNA) molecule and the enzymatic protein is a Cas protein or Cas ortholog.

30. The modified Treg of claim 29, wherein the Cas protein is a Cas9 protein.

31. The modified Treg of claim 29, wherein the Cas protein is a wild-type Cas protein comprising two enzymatically active domains, and capable of inducing double stranded DNA breaks.

32. The modified Treg of claim 29, wherein the Cas protein is a Cas nickase mutant comprising one enzymatically active domain and capable of inducing single stranded DNA breaks.

33. The modified Treg of claim 29, wherein the Cas protein is a deactivated Cas protein (dCas) and is associated with a heterologous protein capable of modulating the expression of the one or more endogenous target genes.

34. The modified Treg of claim 33, wherein the heterologous protein is selected from the group consisting of MAX-interacting protein 1 (MXI1), Krüppel-associated box (KRAB) domain, methyl-CpG binding protein 2 (MECP2), or four concatenated mSin3 domains (SID4X).

35. The modified Treg of any one of claims 24-34, wherein the gene-regulating system is capable of reducing the expression and/or function of a plurality of endogenous target genes selected from the group consisting of TNFRSF4, PRDM1, REEP3, MRPL32, FSCN3, KLC3, C4BPA, LZTS1, CDK16, and ADNP.

36. The modified Treg of claim 35, wherein the gene-regulating system is capable of reducing the expression and/or function of at least 2, 3, 4, 5, 6 or more of endogenous target genes selected from the group consisting of TNFRSF4, PRDM1, REEP3, MRPL32, FSCN3, KLC3, C4BPA, LZTS1, CDK16, and ADNP.

37. The modified Treg of claim 1, wherein the gene-regulating system is capable of reducing the expression and/or function of a plurality of endogenous target genes, wherein at least one of the plurality of target genes is TNFRSF4 and wherein at least one of the plurality of target genes is selected from PRDM1, REEP3, MRPL32, FSCN3, KLC3, C4BPA, LZTS1, CDK16, and ADNP.

38. The modified Treg of claim 37, wherein one of the plurality of target genes is TNFRSF4 and wherein at least 2, 3, 4, 5, 6 or more of the plurality of target genes are selected from PRDM1, REEP3, MRPL32, FSCN3, KLC3, C4BPA, LZTS1, CDK16, and ADNP.

39. The modified Treg of claim 12, wherein the gene-regulating system is capable of reducing the expression and/or function of a plurality of endogenous target genes, wherein at least one of the plurality of target genes is PRDM1 and wherein at least one of the plurality of target genes is selected from TNFRSF4, REEP3, MRPL32, FSCN3, KLC3, C4BPA, LZTS1, CDK16, and ADNP.

40. The modified Treg of claim 39, wherein one of the plurality of target genes is PRDM1 and wherein at least 2, 3, 4, 5, 6 or more of the plurality of target genes are selected from TNFRSF4, REEP3, MRPL32, FSCN3, KLC3, C4BPA, LZTS1, CDK16, and ADNP.

41. The modified Treg of any one of claims 37-40, wherein the gene-regulating system comprises a plurality of gRNA molecules.

42. The modified Treg of any one of claims 1-41, wherein the gene-regulating system is introduced to the Treg by transfection, transduction, electroporation, or physical disruption of the cell membrane by a microfluidics device.

43. The modified Treg of claim 42, wherein the gene-regulating system is introduced as a polynucleotide encoding one or more components of the system, a protein, or a ribonucleoprotein (RNP) complex.

44. The modified Treg of any one of claims 1-43, wherein the immunosuppressive function is selected from Treg proliferation, Treg viability, Treg stability, increased expression or secretion of an immunosuppressive cytokine, optionally wherein the immunosuppressive cytokine is IL-10, increased co-expression of Foxp3 and Helios, and/or resistance to exhaustion.

45. The modified Treg of claim 44, wherein Treg stability is assessed during in vitro culture with IL-6.

46. The modified Treg of any one of claims 1-45, further comprising an engineered immune receptor displayed on the cell surface.

47. The modified Treg of claim 46, wherein the engineered immune receptor is a chimeric antigen receptor (CAR) comprising an antigen-binding domain, a transmembrane domain, and an intracellular signaling domain.

48. The modified Treg of claim 47, wherein the engineered immune receptor is an engineered T cell receptor (TCR).

49. The modified Treg of any one of claims 46-48, wherein the engineered immune receptor specifically binds to an antigen expressed on a target cell.

50. The modified Treg of any one of claims 1-49, wherein the Treg is a human Treg.

51. A modified Treg comprising reduced expression and/or function of one or more endogenous genes relative to the expression and/or function of the one or more endogenous genes in a non-modified Treg, wherein the one more endogenous genes comprises TNFRSF4, and wherein the reduced expression and/or function of the one or more endogenous genes enhances an immunosuppressive function of the Treg.

52. A modified Treg comprising reduced expression and/or function of one or more endogenous genes relative to the expression and/or function of the one or more endogenous genes in a non-modified Treg, wherein the one more endogenous genes comprises PRDM1, and wherein the reduced expression and/or function of the one or more endogenous genes enhances an immunosuppressive function of the Treg.

53. A modified Treg comprising reduced expression and/or function of one or more endogenous genes relative to the expression and/or function of the one or more endogenous genes in a non-modified Treg, wherein the one or more endogenous genes are selected from the group consisting of TNFRSF4, PRDM1, REEP3, MRPL32, FSCN3, KLC3, C4BPA, LZTS1, CDK16, and ADNP, and wherein the reduced expression and/or function of the one or more endogenous genes enhances an immunosuppressive function of the Treg.

54. The modified Treg of any one of claims 51-53 further comprising an engineered immune receptor displayed on the cell surface.

55. The modified Treg of claim 54, wherein the engineered immune receptor is a CAR or an engineered TCR.

56. The modified Treg of claim 54 or 55, wherein the engineered immune receptor specifically binds to an antigen expressed on a target cell.

57. The modified Treg of any one of claims 53-56, further comprising reduced expression of TNFRSF4.

58. The modified Treg of claim 57, comprising reduced expression and/or function of TNFRSF4 and reduced expression and/or function of at least one target gene selected from PRDM1, REEP3, MRPL32, FSCN3, KLC3, C4BPA, LZTS1, CDK16, and ADNP.

59. The modified Treg of claim 58, comprising reduced expression and/or function of TNFRSF4 and reduced expression and/or function of at least 2, 3, 4, 5, 6 or more target genes selected from the group consisting of PRDM1, REEP3, MRPL32, FSCN3, KLC3, C4BPA, LZTS1, CDK16, and ADNP.

60. The modified Treg of any one of claims 53-56, further comprising reduced expression of PRDM1.

61. The modified Treg of claim 60, comprising reduced expression and/or function of PRDM1 and reduced expression and/or function of at least one target gene selected from TNFRSF4, REEP3, MRPL32, FSCN3, KLC3, C4BPA, LZTS1, CDK16, and ADNP.

62. The modified Treg of claim 61, comprising reduced expression and/or function of PRDM1 and reduced expression and/or function of at least 2, 3, 4, 5, 6 or more target genes selected from the group consisting of TNFRSF4, REEP3, MRPL32, FSCN3, KLC3, C4BPA, LZTS1, CDK16, and ADNP.

63. The modified Treg of claim 1 or claim 13, wherein the gene-regulating system comprises a nucleic acid molecule selected from an siRNA and an shRNA.

64. The modified Treg of claim 63, wherein the gene-regulating system is further capable of reducing the expression of one or more endogenous target genes selected from the group consisting of TNFRSF4, PRDM1, REEP3, MRPL32, FSCN3, KLC3, C4BPA, LZTS1, CDK16, and ADNP.

65. The modified Treg of claim 63, wherein the gene-regulating system is capable of reducing the expression and/or function of a plurality of endogenous target genes and comprises a plurality of siRNAs or shRNAs, wherein at least one endogenous target gene is selected from the group consisting of TNFRSF4, PRDM1, REEP3, MRPL32, FSCN3, KLC3, C4BPA, LZTS1, CDK16, and ADNP.

66. The modified Treg of claim 65, wherein the gene-regulating system is capable of reducing the expression and/or function of at least 2, 3, 4, 5, 6 or more of endogenous target genes selected from the group consisting of TNFRSF4, PRDM1, REEP3, MRPL32, FSCN3, KLC3, C4BPA, LZTS1, CDK16, and ADNP.

67. The modified Treg of claim 63, wherein the gene-regulating system is capable of reducing the expression and/or function of a plurality of endogenous target genes and comprises a plurality of siRNAs or shRNAs, wherein at least one of the plurality of target genes is TNFRSF4 and at least one of the plurality of target genes is selected from PRDM1, REEP3, MRPL32, FSCN3, KLC3, C4BPA, LZTS1, CDK16, and ADNP2.

68. The modified Treg of claim 67, wherein at least one of the plurality of target genes is TNFRSF4 and at least at least 2, 3, 4, 5, 6 or more of the plurality of target genes are selected from PRDM1, REEP3, MRPL32, FSCN3, KLC3, C4BPA, LZTS1, CDK16, and ADNP.

69. The modified Treg of claim 63, wherein the gene-regulating system is capable of reducing the expression and/or function of a plurality of endogenous target genes and comprises a plurality of siRNAs or shRNAs, wherein at least one of the plurality of target genes is PRDM1 and at least one of the plurality of target genes is selected from TNFRSF4, REEP3, MRPL32, FSCN3, KLC3, C4BPA, LZTS1, CDK16, and ADNP2.

70. The modified Treg of claim 69, wherein at least one of the plurality of target genes is PRDM1 and at least at least 2, 3, 4, 5, 6 or more of the plurality of target genes are selected from TNFRSF4, REEP3, MRPL32, FSCN3, KLC3, C4BPA, LZTS1, CDK16, and ADNP.

71. The modified Treg of any one of claims 51-70, wherein the Treg is a human Teg.

72. A composition comprising the modified Tregs of any one of claims 1-71.

73. The composition of claim 72, wherein the composition comprises at least 1×10⁴, 1×10⁵, 1×10⁶, 1×10⁷, 1×10⁸, 1×10⁹, or 1×10¹⁰ modified Tregs.

74. The composition of claim 72 or 73, suitable for administration to a subject in need thereof.

75. The composition of any one of claims 72-74, comprising autologous Tregs derived from the subject in need thereof.

76. The composition of any one of claims 72-74, comprising allogeneic Tregs derived from a donor subject.

77. A gene-regulating system capable of reducing expression of one or more endogenous target genes in a cell, wherein the system comprises (i) a nucleic acid molecule; (ii) an enzymatic protein; or (iii) a nucleic acid molecule and an enzymatic protein, and wherein the one or more endogenous target genes comprises TNFRSF4.

78. The gene-regulating system of claim 77, wherein the system comprises a guide RNA (gRNA) nucleic acid molecule and a Cas endonuclease.

79. A gene-regulating system capable of reducing expression of one or more endogenous target genes in a cell, wherein the system comprises (i) a nucleic acid molecule; (ii) an enzymatic protein; or (iii) a nucleic acid molecule and an enzymatic protein, and wherein the one or more endogenous target genes comprises PRDM1.

80. The gene-regulating system of claim 79, wherein the system comprises a guide RNA (gRNA) nucleic acid molecule and a Cas endonuclease.

81. A gene-regulating system capable of reducing expression and/or function of one or more endogenous target genes in a cell, wherein the system comprises (i) a nucleic acid molecule; (ii) an enzymatic protein; or (iii) a nucleic acid molecule and an enzymatic protein, and wherein the one or more endogenous target genes are selected from the group consisting REEP3, MRPL32, FSCN3, KLC3, C4BPA, LZTS1, CDK16, and ADNP2.

82. The gene-regulating system of claim 81, wherein the system comprises a guide RNA (gRNA) nucleic acid molecule and a Cas endonuclease.

83. The gene-regulating system of any one of claims 78-82, wherein the Cas protein is a Cas9 protein.

84. The gene-regulating system of any one of claims 78-82, wherein the Cas protein is a wild-type Cas protein comprising two enzymatically active domains, and capable of inducing double stranded DNA breaks.

85. The gene-regulating system of any one of claims 78-82, wherein the Cas protein is a Cas nickase mutant comprising one enzymatically active domain and capable of inducing single stranded DNA breaks.

86. The gene-regulating system of any one of claims 78-82, wherein the Cas protein is a deactivated Cas protein (dCas) and is associated with a heterologous protein capable of modulating the expression of the one or more endogenous target genes.

87. The gene-regulating system of claim 86, wherein the heterologous protein is selected from the group consisting of MAX-interacting protein 1 (MXI1), Krüppel-associated box (KRAB) domain, and four concatenated mSin3 domains (SID4X).

88. The gene-regulating system of claims 77, 79 or 81, wherein the system comprises a nucleic acid molecule and wherein the nucleic acid molecule is an siRNA, an shRNA, a microRNA (miR), an antagomiR, or an antisense RNA.

89. The gene-regulating system of claims 77, 79 or 81, wherein the system comprises a protein comprising a DNA binding domain and an enzymatic domain and is selected from a zinc finger nuclease and a transcription-activator-like effector nuclease (TALEN).

90. A kit comprising the gene-regulating system of any one of claims 77-89.

91. A gRNA nucleic acid molecule comprising a targeting domain nucleic acid sequence that is complementary to a target sequence in an endogenous target gene, wherein the endogenous target gene is TNFRSF4.

92. A gRNA nucleic acid molecule comprising a targeting domain nucleic acid sequence that is complementary to a target sequence in an endogenous target gene, wherein the endogenous target gene is PRDM1.

93. A gRNA nucleic acid molecule comprising a targeting domain nucleic acid sequence that is complementary to a target sequence in an endogenous target gene, wherein the endogenous target gene is selected from REEP3, MRPL32, FSCN3, KLC3, C4BPA, LZTS1, CDK16, and ADNP2.

94. The gRNA molecule of any one of claims 91-93, wherein the target sequence comprises a PAM sequence.

95. The gRNA molecule of any one of claims 91-94, wherein the gRNA is a modular gRNA molecule.

96. The gRNA molecule of any one of claims 91-94, wherein the gRNA is a dual gRNA molecule.

97. The gRNA molecule of any one of claims 91-96, wherein the targeting domain is 16, 17, 18, 19, 20, 21, 22, 23, 24, 25, 26 or more nucleotides in length.

98. The gRNA molecule of any one of claims 91-97, comprising a modification at or near its 5′ end (e.g., within 1-10, 1-5, or 1-2 nucleotides of its 5′ end) and/or a modification at or near its 3′ end (e.g., within 1-10, 1-5, or 1-2 nucleotides of its 3′ end).

99. The gRNA molecule of claim 98, wherein the modified gRNA exhibits increased stability towards nucleases when introduced into a T cell.

100. The gRNA molecule of claim 98 or 99, wherein the modified gRNA exhibits a reduced innate immune response when introduced into a T cell.

101. A polynucleotide molecule encoding the gRNA molecule of any one of claims 91-100.

102. A polynucleotide molecule encoding a plurality of gRNA molecules selected from any one of claims 91-100.

103. A composition comprising one or more gRNA molecules according to any one of claims 91-100 or the polynucleotide of claim 101 or 102.

104. A kit comprising the gRNA molecule of any one of claims 91-100 or the polynucleotide of claim 101 or 102.

105. A method of producing a modified Treg comprising:
obtaining an Treg from a subject;
introducing a gene-regulating system into the Treg, wherein the gene-regulating system is capable of reducing expression and/or function of one or more endogenous target genes, and wherein the one or more endogenous target genes comprises TNFRSF4; and
culturing the Treg such that the expression and/or function of one or more endogenous target genes is reduced compared to a Treg that has not been modified.

106. A method of producing a modified Treg comprising:
obtaining a Treg from a subject;
introducing a gene-regulating system into the Treg, wherein the gene-regulating system is capable of reducing expression and/or function of one or more endogenous target genes, and wherein the one or more endogenous target genes comprises PRDM1; and
culturing the Treg such that the expression and/or function of one or more endogenous target genes is reduced compared to an Treg that has not been modified.

107. A method of producing a modified Treg comprising:
introducing a gene-regulating system into the Treg, wherein the gene-regulating system is capable of reducing expression and/or function of one or more endogenous target genes, wherein the one or more endogenous target genes comprises TNFRSF4.

108. A method of producing a modified Treg comprising:
introducing a gene-regulating system into the Treg, wherein the gene-regulating system is capable of reducing expression and/or function of one or more endogenous target genes, wherein the one or more endogenous target genes comprises PRDM1.

109. The method of any one of claims 105-108, wherein the gene-regulating system is one selected from claims 74-86.

110. The method of any one of claims 105-108, further comprising introducing a polynucleotide sequence encoding an engineered immune receptor selected from a CAR and a TCR.

111. The method of claim 110, wherein the gene-regulating system and/or the polynucleotide encoding the engineered immune receptor are introduced to the Treg by transfection, transduction, electroporation, or physical disruption of the cell membrane by a microfluidics device.

112. The method of any one of claims 107-111, wherein the gene-regulating system is introduced as a polynucleotide sequence encoding one or more components of the system, as a protein, or as a ribonucleoprotein (RNP) complex.

113. A method of producing a modified Treg comprising:
obtaining a population of Tregs;
expanding the population of Tregs; and
introducing a gene-regulating system into the population of Tregs, wherein the gene-regulating system is capable of reducing expression and/or function of one or more endogenous target genes comprising TNFRSF4.

114. The method of claim 113, wherein the gene-regulating system is introduced to the population of Tregs prior to the expansion.

115. The method of claim 113, wherein the gene-regulating system is introduced to the population of Tregs after the expansion.

116. A method of producing a modified Treg comprising:
obtaining a population of Tregs;
expanding the population of Tregs; and
introducing a gene-regulating system into the population of Tregs, wherein the gene-regulating system is capable of reducing expression and/or function of one or more endogenous target genes comprising PRDM1.

117. The method of claim 113, wherein the gene-regulating system is introduced to the population of Tregs prior to the expansion.

118. The method of claim 113, wherein the gene-regulating system is introduced to the population of Tregs after the expansion.

119. The method of any one of claims 105-118, wherein the Treg is a human Treg.

120. A method of treating a disease or disorder in a subject in need thereof comprising administering an effective amount of the modified Tregs of any one of claims 1-71, or the composition of any one of claims 72-76.

121. The method of claim 120, wherein the disease or disorder is an autoimmune disorder.

122. The method of claim 121, wherein the autoimmune disorder is autoimmune hepatitis, inflammatory bowel disease (IBD), Crohn's disease, colitis, ulcerative colitis, type 1 diabetes, alopecia areata, vasculitis, temporal arthritis, lupus, celiac disease, Sjogrens syndrome, polymyalgia rheumatica, multiple sclerosis, arthritis, rheumatoid arthritis, graft versus host disease (GVHD), and psoriasis.

123. The method of any one of claims 120-122, wherein the modified Tregs are autologous to the subject.

124. The method of any one of claims 120-122, wherein the modified Tregs are allogeneic to the subject.

125. A method of enhancing one or more immunosuppressive functions of a Treg comprising:
introducing a gene-regulating system into the Treg, wherein the gene-regulating system is capable of reducing the expression and/or function of one or more endogenous target genes, and wherein the one or more endogenous target genes comprises TNFRSF4; and
culturing the Treg such that the expression and/or function of one or more endogenous target genes is reduced compared to an Treg that has not been modified,
wherein the modified Treg demonstrates one or more enhanced immunosuppressive functions compared to the Treg that has not been modified.

126. A method of enhancing one or more immunosuppressive functions of a Treg comprising:
introducing a gene-regulating system into the Treg, wherein the gene-regulating system is capable of reducing the expression and/or function of one or more endogenous target genes, and wherein the one or more endogenous target genes comprises PRDM1; and
culturing the Treg such that the expression and/or function of one or more endogenous target genes is reduced compared to an Treg that has not been modified,
wherein the modified Treg demonstrates one or more enhanced immunosuppressive functions compared to the Treg that has not been modified.

127. A method of enhancing one or more immunosuppressive functions of an Treg comprising:
introducing a gene-regulating system into the Treg, wherein the gene-regulating system is capable of reducing the expression and/or function of one or more endogenous target genes, wherein the one or more endogenous target genes comprises TNFRSF4.

128. A method of enhancing one or more immunosuppressive functions of an Treg comprising:
introducing a gene-regulating system into the Treg, wherein the gene-regulating system is capable of reducing the expression and/or function of one or more endogenous target genes, wherein the one or more endogenous target genes comprises PRDM1.

129. The method of any one of claims 125-128, wherein the one or more immunosuppressive function is selected from Treg proliferation, Treg viability, Treg stability, increased expression or secretion of an immunosuppressive cytokine, optionally wherein the immunosuppressive cytokine is IL-10, increased co-expression of Foxp3 and Helios, and/or resistance to exhaustion.

130. The method of claim 129, wherein Treg stability is assessed during in vitro culture with IL-6.

131. A method of treating an autoimmune disease in a subject in need thereof comprising administering an effective amount of a modified Treg of any one of claims 1-71, or the composition of any one of claims 72-76.

132. The method of claim 131, wherein the autoimmune disease is selected from the group consisting of: autoimmune hepatitis, inflammatory bowel disease (IBD), Crohn's disease, colitis, ulcerative colitis, type 1 diabetes, alopecia areata, vasculitis, temporal arthritis, lupus, celiac disease, Sjogrens syndrome, polymyalgia rheumatica, multiple sclerosis, arthritis, rheumatoid arthritis, graft versus host disease (GVHD), and psoriasis.

133. The modified Treg of any one of claims 1-71, wherein the modified Treg is a tissue-resident Treg.

134. The method of any one of claims 105-132, wherein the Treg is a tissue-resident Treg.
